SEQUENCE LISTING (22 sequences)

SEQ ID NO: 1 - protein, 2700 aa, Ralstonia solanacearum
MTTHALTERATLVDWIEHHARARPLAEALFFCGHGADDLRLGYGALSERVRRCAAALQQRGAAGSTALILFPSGIDYVVALLACFYAGVTGVPVNLPGVSRVRRVLPKLGDITRDCRPAVVLTHTAIERASGNDLRDFAAGHGLDILHLDTLGGEAAAWVRPALTPESIAFLQYTSGSTGSPKGVVNRHGALLRNLQFLGRLTRPQDRAPEDTAVASWLPLFHDLGLIMGILLPLAYGNRAVYMAPMAFVADPLRWLEIATAERATALPCPSFALRLCADEARRAAPARTAGIDLSSVQCLMPAAEPVLPSQIEAFQAAFAAHGMRREAIRPAYGLAEATLLVSANVDDAPPHRIDVETAPLEQGRAVVHPAAAPMPAAGRRRYVSNGREFDGQDVRIVDPRTCATLPEGTVGEIWISGPCIAGGYWNKAELNREIFMAETPGAGDRRYLRTGDMGFLHGGHLFVTGRLKDMMLFRGQCHYPNDIEATSGRAHAAAIPESGAAFSIQAEDEAGERLVIVQEVRKQAGIDPRDIATAVRAAVAEGHALGVHAVVLIRKGTLPRTTSGKVRRAAVREAWLAGTLQTLWQDDIDNLAVPPTPAQETAAAPADAALLAALAPLDAARRQQHLVQWLAARAAAALGTVAARAIRPEASLFGYGLDSMSATRLAAVAAAASGLALPDSLLFDHPSLDGLAGWLLQAMEQARHLPPAPGGRDRAMPAPRPAAHRHGDGQDPIAIIGMAFRLPGENGHDADTDAAFWRLLDGAGCAIRPMPAERFRAPAGMPGFGAYLNQVDRFDAAFFGMSPREAMNTDPQQRLLLEVAWHALEDAGLPPGDLRGSDSGVFVGIGTADYGHLPFISGDDAHFDAYWGTGTSFAAACGRLSFTFGWEGPSMAVDTACSASHSALHLAVQALRARECGMALSAGVKLQLLPEIDRVLHKAGMLAADGRCKTLDASADGYVRGEGCVVLVLKRLSDALADGDAIRAVIRDTLVRQDGAGSSLSAPNGEAQQRLLSLALARAGLAPSEIDYIELHGTGTRLGDPIEYQSVADVFGGRAPDDPLWIGSVKTNIGHLESAAGAAGLVKTVLALEQARIPPLVGLKGINPLIDLDAIPARAPAHTVDWPARQAVRRAGVTSYGFAGTIAHVILEQAPQAPVAQAAGTEPTRGPHLFLLSARSPDALRRLAAAYRDTLAGTADLAVLANGMARQREHHALRAAVVASDHDECARALDRLAAPDAAAPEAVTRAPRVGFLFTGQGSQYAGMTRALYAAQPDFRAALDAADAALAPHLGRSILALMHDDAQRDALQQTAHAQPALFACGYALAAMWQAWGVVPAVLVGHSIGEFAAMVVAGAMTLEDAARLIVRRGALMQALPAGGAMLAARATPRHAHDLLAALAPAVAAEVSLAAINGPQDVVFSGSAAGIDAVRARLDAQQLDARPLAVSHAFHSPLLDPMLGDWAEACADAQSAPPRIPLISTLTGAPMTTAPDAAYWSAHARQPVRFAEALARAGADCDVLLEIGAHAVLSALAQRNQLAQPWPHPVACVASLLRGTDDSRAVAQACAELYLRGQPFDWDRLFAGPLPSPRALPRYPFDRQSHWLEYDEDAPRTPLPMQPQPERAAPRPVERYAVQWEPFAPSAGDGHASTYWIVAADAADAGPADAGRLAARLSGPARDVHVLSPSQWADAADRIADDDVVIYLAGWPARASDAAAVAGSRHVWQLTECVRTLQRLRKTPRILLPTLHGQSPDGAPCDPLQAALWGAARPLSLEYPGPAWLLADCAGESPLETLADALPALLPLFGKEEAVALRAGGWLRPRLTPQAAPERAPCVTLRADGLYLVAGAYGALGRHTTDWLAAHGATHLVLAGRRAPPAGWQARLALLRAQGVRIDPVDADLAEAADVERLFDAVAALEATTGRTLAGVFHCAGTSRFNDLAGLTTDDCAAVTGAKMTGAWLLHEQTRARRLDWFVCFTSISGVWGSRLQIPYGAANAFQDALVRLRRAQGLPALAVAWGPWGGGAGMSEVDDALLQLLRAAGIRRLAPSRYLATLDHLLGHAEHADGLPADGTCVVAEVDWQQFIPLFALYNPIGTFERCRTDTATHATAAPSALIALDSGARADAVRAFVIAELARTLRVAPSQLTPDIELLKLGMDSILVMDFSRRCESGLGVKCELKAIFERNTPGGLASYLLERLEHAPQGAVPAPAAAEPIVHAPDHAHLPFPLTELQHAYWIGRQGHYALGGVACHAYLEADAADGLDLGLLERCWNALVARHGALRLVIDESGQQRILPRVPAYRIRVANLGAATPQALAAHCDDWRQAMSHQVLDAAQWPLFDVRATHLPGGATRLHIGIDMLINDATSGQIIWDELAALYRAGGDLERAGLAPFEISFRDYVLAKYVHSEARRAARESAKAYWLGQLETLPPAPQLPLRAEALHRAAPRFSRRQHRLSAPQWQSLRDRAAASGCTPASLLIAVFAEVLSAWSTEPRFTLNLTTFDRLPWHADVPRLLGDFTAVTLLPLDCAAPLPFGQRAAAVNGAVLEHLQHRAFSAVDVLREWNRGRERQDAVSMPVVFTSQLGMSDPTKGAARASVLGTVGYGISQTPQVWLDHQACELDGALIYNWDAVDALFQPGVLDAMFDAYNRMLERLAADADAWLEPLPALLPQAQREVRARVNASTAPLPERCLDQLFFDQA

SEQ ID NO: 2 - protein, 832 aa, Ralstonia solanacearum
MMTITTDRTPPAAGAALDRNRSAYAGLADVLERAGLAEHALYLNWGYRPVDGQPDWAARELPPGELGRMQARLVLEVLGDTPLDGRRVLDVGCGRGGALALMGRLHAPAALAGADISAANIAYCRKRHTHPRLRFQIADACRLPYPDSSMDVVFNLESSGAYPDIGAFFHHVHRILRVGGRFCLADVFDADSVAWVRAALEQAGFTLERERSIPAQVRAARERASPGIWRRLDTALTALDAPGLRRELERYLAAPSSGLFQALEDGRVDYRLFHWRKTCPAAGRIDADVIARLATRSARLDAALQDRAPSAAAPQSPAPGPANASASAWFPFTAPDAQAGFNVFALPYAGGGASVYRAWTLPRRPGAAPWQLCPVQLPGRESRFGEPLIDDMATLADRLADAIGPYAHRPWALLGCSLGCKIAFEVARRFARQGRPPALLFLMACPAPGLPLGRRISTRAEADFAREVCHLGGTPPEVLADAEMMRTLMPILRNDSALAEHYVAAEDATVNVPIVMVAAGDDHLVTVEEARRWQRHAGAGFDWRLVDGGHFFLRQRRRELTDWLLDALRRGERTLPVQTTTTDVPDVPCSTPEQPRDPSRMPAPGASANLVLAPGEILVVTAPRSLAARLTPAVLSDDEQRQLARFAFDADRERYLAAHWAKRRVLGALLAAAPRSLRFGAQAGGKPYLIGEALHFSLSHSGDRVAVAVCRHAPVGVDIEQARGIACHASAARIMHPLDRIAPQCETPEDRFLAAWSLKEAVAKCTGAGLALPFDSLRLAFAGNGRYGCLLGTHAAWEAHHQHEDGVHLAVASATPWAALRILPLDAALAEG

SEQ ID NO: 3 - protein, 394 aa, Streptomyces sp.
MAGRTATRRITLFDPERFRCRIAAECDFDAAALGLTPQEIRRMDRAVQMAVAATGEALADAGVGEGDLDPARTGVTIGNAVGSTMMMEEEYVVISDGGRKWLCDEEYGVRHLYGAVIPSTAGVEVARRVGAEGPTAVVSTGCTSGLDAVGHAAQLIEEGSADVVIGGATDAPISPITVACFDSLKATSTRNDDAEHACRPFDRDRDGLVLGEGSAVFVMEARERAVRRGAKIYCEVAGYAGRANAYHMTGLKPDGRELAEAIDRAMAQAGISAEDIDYVNAHGSGTRQNDRHETAAFKRSLRDHARRVPVSSIKSMVGHSLGAIGAIEVAASALAIEHGVVPPTANLTTPDPECDLDYVPREAREHPTDVVLSVGSGFGGFQSAVVLISPRSRR

SEQ ID NO: 4 - protein, 336 aa, Streptomyces sp.
MSQLSLSQAAPAGGSRIRGVGAYRPARVVTNEEIAPRIGVAPEWIARRSGIHTRRFAGPDEPLAMMAATASEKALAAAGLSADEVDCVLVATISHLLQMPALAVDVAHRLGAAPTAAFDLSAACAGFCHGVAIADSMVRSGTAHNVLLVGADRMTDVVDADDPATAFLFADGAGAVVIGPSETPGIGPVAWGSDGERMDAITMTGHWTPSLRTNPELPWPYLCMTGWKVFRWATETMGQAARDAIERAGVTSEELSAFIPHQANGLITDALAKDIGLTADTAIARDITDSGNTSGASIPMAMERLLASGQARSGEAALLIGFGSGLVHAGQVVLLP

SEQ ID NO: 5 - protein, 579 aa, Streptomyces sp.
MELALPAELAPTLPEALRLRSEQQPDTVAYVFLRDGETPEETLTYGRLDRAARARAAALEAAGLAGGTAVLLYPSGLEFVAALLGCMYAGTAGAPVQVPTRRRGMERARRIADDAGAKTILTTTAVKREVEEHFADLLTGLTVIDTESLPDVPDDAPAVRLPGPDDVALLQYTSGSTGDPKGVEVTHANFRANVAETVELWPVRSDGTVVNWLPLFHDMGLMFGVVMPLFTGVPAYLMAPQSFIRRPARWLEAISRFRGTHAAAPSFAYELCVRSVADTGLPAGLDLSSWRVAVNGAEPVRWTAVADFTEAYAPAGFRPQAMCPGYGLAENTLKLSGSPEDRPPTLLRADAAALQDGRVVPLTGPGTDGVRLVGSGVTVPSSRVAVVDPGTGTEQPAGRVGEIWINGPCVARGYHGRPAESAESFGARIAGQEARGTWLRTGDLGFLHDGEVFVAGRLKDVVIHQGRNFYPQDIELSAEVSDRALHPNCAAAFALDDGRTERLVLLVEADGRALRNGGADALRARVHDAVWDRQRLRIDEIVLLRRGALPKTSSGKVQRRLARSRYLDGEFGPAPAREA

SEQ ID NO: 6 - protein, 543 aa, Cannabis sativa
MEKSGYGRDGIYRSLRPPLHLPNNNNLSMVSFLFRNSSSYPQKPALIDSETNQILSFSHFKSTVIKVSHGFLNLGIKKNDVVLIYAPNSIHFPVCFLGIIASGAIATTSNPLYTVSELSKQVKDSNPKLIITVPQLLEKVKGFNLPTILIGPDSEQESSSDKVMTFNDLVNLGGSSGSEFPIVDDFKQSDTAALLYSSGTTGMSKGVVLTHKNFIASSLMVTMEQDLVGEMDNVFLCFLPMFHVFGLAIITYAQLQRGNTVISMARFDLEKMLKDVEKYKVTHLWVVPPVILALSKNSMVKKFNLSSIKYIGSGAAPLGKDLMEECSKVVPYGIVAQGYGMTETCGIVSMEDIRGGKRNSGSAGMLASGVEAQIVSVDTLKPLPPNQLGEIWVKGPNMMQGYFNNPQATKLTIDKKGWVHTGDLGYFDEDGHLYVVDRIKELIKYKGFQVAPAELEGLLVSHPEILDAVVIPFPDAEAGEVPVAYVVRSPNSSLTENDVKKFIAGQVASFKRLRKVTFINSVPKSASGKILRRELIQKVRSNM

SEQ ID NO: 7 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 8 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 9 - protein, 99 aa, Cannabis sativa
AVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPR

SEQ ID NO: 10 - protein, 95 aa, Cannabis sativa
AVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFD

SEQ ID NO: 11 - protein, 415 aa, Ralstonia solanacearum
MAFNERVVDWQQVAGAQPDASPERMSADDPFMIIYTSGTTGKPKGTVHTHGSFPMKIAHDSAIHFNVSPKDVFCWPADMGWVAGTLVMSCALLRGATLVCYDGAPDFPDWSRMSRLIERHRVTHFGSAPTLIRGLASNEAIATQGDVSSVKLLITAGEGIDPEHFLWFQKAFGGGHRPVINYTGGTEVSGALLSSVVIKPISPAGFNTASPGVATDVVDAEGHSVTGEVGELAIRKPFIGMTRSFWQDDERYLDSYWRTIPGIWVHGDLAMRREDGMWFMMGRSDDTIKLAGKRLGPAEIEDVLLELPEIAEAAAIGVEDPVKGQKLVVFVVASKASTASADALASVIGKHVDLRLGRPFRPSVVHVVAQLPKTRSSKIMRRVIRSVYTGKPAGDLSSLDNPLALDEIRSAAAVS

SEQ ID NO: 12 - protein, 109 aa, Arabidopsis thaliana
MEEAKGPVKHVLLASFKDGVSPEKIEELIKGYANLVNLIEPMKAFHWGKDVSIENLHQGYTHIFESTFESKEAVAEYIAHPAHVEFATIFLGSLDKVLVIDYKPTSVSL

SEQ ID NO: 13 - protein, 146 aa, Streptomyces sp.
AGRTDNSVVIDAPVQLVWDMTNDVSQWAVLFEEYAESEVLAVDGDTVRFRLTTQPDEDGKQWSWVSERTRDLENRTVTARRLDNGLFEYMNIRWEYTEGPDGVRMRWIQEFSMKPSAPVDDSGAEDHLNRQTVKEMARIKKLIEEA

SEQ ID NO: 14 - protein, 216 aa, Aspergillus nidulans
MAPNHVLFFPQERVTFDAVHDLNVRSKSRRRLQSLLAAASNVVQHWTASLDGLERADIFSFEDLVELAERQTTQTRGSIVADLVLLTTVQIGQLLVLAEDDPAILSGHAGARAIPMGFGAGLVAAGVAAAATSADGIVNLGLEAVSVAFRLGVELQRRGKDIEDSNGPWAQVISSATTIADLEQALDRINASLRPINQAYIGEVMTESTVVFGPPS

SEQ ID NO: 15 - protein, 2373 aa, Fusarium graminearum
MAARRVVLFGGQGSRSIFSSSTTSIAEQDAQSSTAGIILLSKCHVAILREISSLDVQSRLILAIDPVSFPTPRHLLQIADKYHTHPVLQATTIYLCQILRYLSHTLQQDDTFEQCFERIEATAGFSSGIIPAAVVACSSTIDEFVVCAVEGFRLAFWVAYYSFRWSLLLAEQNGHNTSQDATMSLATRGLSRTQVEQVLYRMKAERGLQRMAISSIAISGSVSISGPQAELVALQGELQSLRYVTTTFAYVHGWYHGGKQLEPVVKQVEETINRRCICFPSCDGSSKPIYSTLDGTVLDLFGGSSNKPLSSLTRHLLIHCVNWRDTSRAIAADIREILRHTPMAVDILSFGPASSSIFPTIDSQNPRVNLVDMSSFKSQEGSTTQHLDRPNDIAIVGMSTNLPGGHNAAQLWETLSSGLNTVQEIPESRFQISDYYTSEKGEPRSMATGHGAFLDDPFSFDNAFFNISPREAKSMDPQQRILLHGAQEALEDAGYVADSTPSSQRATTGCYIGLATGDYTDNLHDDIDAFYPSGTLRAFHSGRISYFYQLSGPSIVTDTACSSSTVSIYQACRAIQNGDCTTAIAGGVNVITSPDMYLSLSRGHFLSPTGNCKPFDASADGYCRAEGCVLFVLKRLSDAVAEGDRIHAVIRNAQINQSGNSSSITHPHSPTQTDLLTRLLKQADVDPASISVVEAHGTGTQAGDAREIETLKLVFSQYHSATTPLVVSSIKGNVGHCEAASGAAGLAKLLLMLRNDEIPKQAGLENMNPALGDLQNSGLVVPRQNMPWNRSRTVPRRAVLNNFGAAGSNASLLLEEWLESPATSKQKNEEGKRSSYVFALSAKSNKALQLSVGRHIETLKKNMELGTSLEDICYTATARRQQFDHRISATCSSKLELMDKLEQYQSTVSTPAQMVSSTVFIFTGQGSIYSGMGRELMSTYPPFRDIIRTCDRIVQGLGLGCPSILNYILPGTEGRLASMSHVEHLMVSQCACVALEYALAKTFISWGIKPDYVMGHSLGEYTALCISGVLTPGDTFRLVATRAKMMGEHCAANTSGMLACHLSSGEIQSIISDDPSFCQLSIACLNGPHDCVVGGPLTQLEALRTRCKTGNIKCKLIDVPYAFHTSAMDPVLGLLSALGRSVEFQDATIPVISNVDGQLFRKDMTANYFANHTRRPVRFHESIMNLQDLIGQSSLDESLFIEIGPQPAMLPMLRDSIASASCTYLSTLQKGRDAWMSISETLSAISLRKMGINWREVFDGTSAQVTDLPGHPLQGTRFCIPFKEPRGITNHAKSSAIAFATSVRTGCRLLPWVRADTNLSKEHIFETDMTTLGPLISGHDVGGSPICPASVFHELALEAAKSVLEPGKEDILVVKGMKFSSPLIFLSSTSNTTVHVHISKKGIATTRTASFHVKSTSPASPVESLHCSGYVTLQNLEQQSGQWMRDHALVTRQARLFSGAGKDLLSTFRRRVLYENIFTRVVRYSRDYQTLQFLDVADSNLEGMGSFNMPSDSIAQTETAYIAHPVFTDTLLHAAGFIANLAIGSNEVGICSAVESIEVAYHEINYEDTFKIYCSLLEVKGLIVADSFALDSSDNIVAVIRGMEFKKLQLSTFQQALSRISSNSEPEGPEYHHGVSSSAELQLQTSVAACQPLTVDTAIDAHKHQDENGISQILKDVVVEVGGFMEQDIDYTMSLTSLGIDSLMQIEIVSKISRLFPEKTGLDHNALAECETLQELNDMLSSVLQPSVKQRSASQASSSKQTAVITPTSSDSSVEGDSAHGSVVLPVALHTSDESRTPLCLFHDGSGQISMYKRLQGHDRTTYAFFDPKFECSDEGRSFYSSIEDMAEDYASRILSTRPPLSSLILCGWSFGGIVALEVARLLFLRGIEVRGLVLIDSPSPINHEPLPAQIISSITRFTGRSESTNALEEEFLSNASLLGRYKPESLSLTTGRTLKTVMLQSKGTLDTESLCGVRYDWLSRQDVRDAAIAEWESLMTRSPKRHHNFGKHANTSNSLTDKSSASNKAHISMHQRIDLHCHAVAPSYRQYAIDNGHEKPDGMPALPQWTPEQHIGLMKKLNISKSVLSITSPGTHLTPQNDENATRLTRQVNEELSTICQKHPSYFSFFASLPLPSVNDSIAEIDYALDQLGALGFAVLSNANGVYLGDAELDPVFAHLNARKAILFIHPTTCNIIASSGQVQPVKPLEKYPRPMMEFMFDETRAIANLLLSGTVAKYPDIKFIMSHCGCALPSMLDRIGAFATLISGAESQTAEFQRLLRERFYFDLAGFPLPNAIHGLLRILGEGAEKRLVYGTDYPFTPERLVVSLADVMEKGLEELFDEGQRADVLVRVAGTIQDEAMRTTNTEDHSGTLS

SEQ ID NO: 16 - protein, 423 aa, Streptomyces sp.
MSSERRAVITGMGVIAPGGVGTRAFWSAVTAGRTATRRITLFDPERFRCRIAAECDFDAAALGLTPQEIRRMDRAVQMAVAATGEALADAGVGEGDLDPARTGVTIGNAVGSTMMMEEEYVVISDGGRKWLCDEEYGVRHLYGAVIPSTAGVEVARRVGAEGPTAVVSTGCTSGLDAVGHAAQLIEEGSADVVIGGATDAPISPITVACFDSLKATSTRNDDAEHACRPFDRDRDGLVLGEGSAVFVMEARERAVRRGAKIYCEVAGYAGRANAYHMTGLKPDGRELAEAIDRAMAQAGISAEDIDYVNAHGSGTRQNDRHETAAFKRSLRDHARRVPVSSIKSMVGHSLGAIGAIEVAASALAIEHGVVPPTANLTTPDPECDLDYVPREAREHPTDVVLSVGSGFGGFQSAVVLISPRSRR

SEQ ID NO: 17 - protein, 409 aa, Streptomyces sp.
MTVITGLGVVAPTGVGLDDYWATTLAGKSGIDRIRRFDPSGYTAQLAGQVDDFEATDHVPSKLLAQTDRMTHFAFAGANMALADAHVDLADFPEYERAVVTANSSGGVEYGQHELQKMWSGGPMRVSAYMSVAWFYAATTGQLSIHHGLRGPCGLIATEQAGGLDALGHARRLLRRGARIAVTGGTDAPLSPASMVAQLATGLLSSNPDPTAAYLPFDDRAAGYVPGEGGAIMIMEPAEHALRRGAERIYGEIAGYAATFDPAPGTGRGPTLGRAIRNALDDARIAPSEVDLVFADGSGTPAMDRAEAEALTEVFGPRGVPVTVPKAATGRMYSGGGALDVATALLAMRDGVAPPTPHVTELASDCPLDLVRTEPRELPIRHALVCARGVGGFNAALVLRRGDLTTPEH

SEQ ID NO: 18 - protein, 86 aa, Streptomyces sp.
MSTLSVEKLLEIMRATQGESADTSGLTEDVLDKPFTDLNVDSLAVLEVVTQIQDEFKLRIPDSAMEGMETPRQVLDYVNERLEEAA

SEQ ID NO: 19 - protein, 279 aa, Streptomyces sp.
MAGRTDNSVVIDAPVQLVWDMTNDVSQWAVLFEEYAESEVLAVDGDTVRFRLTTQPDEDGKQWSWVSERTRDLENRTVTARRLDNGLFEYMNIRWEYTEGPDGVRMRWIQEFSMKPSAPVDDSGAEDHLNRQTVKEMARIKKLIEEAAARAGVDGGIPAEGKDSVRDATGNGDPGPVFRVLLRAEIADGKEKEFEDAWREIGQVITGQPANLGQWLMRSHDEPGVYYIISDWTDEERFRAFERSEEHVGHRSTLQPFRTKGSMVTTDVVAAMTKAGQTW

SEQ ID NO: 20 - protein, 2103 aa, Aspergillus nidulans
MAPNHVLFFPQERVTFDAVHDLNVRSKSRRRLQSLLAAASNVVQHWTASLDGLERADIFSFEDLVELAERQTTQTRGSIVADLVLLTTVQIGQLLVLAEDDPAILSGHAGARAIPMGFGAGLVAAGVAAAATSADGIVNLGLEAVSVAFRLGVELQRRGKDIEDSNGPWAQVISSATTIADLEQALDRINASLRPINQAYIGEVMTESTVVFGPPSTLDALAKRPELAHATITSPASALAQVPLHGAHLPPISATMIAASSSQQATELWKLAVEEVANKPIDVHQAVTALIHDLHRANITDIVLTAIGASTETSGIQSLLEKNGLAVELGQLSPTPRPYGNDLDSIPADAIAVVGMSGRFPNSDTLDEFWRLLETATTTHQVIPESRFNVDDFYDPTRAKHNALLARYGCFLKNPGDFDHRLFNISPREAMQMDPVQRMLLMTTYEALEMAGYSPPTPAAPGDSEQAPPRIATYFGQTIDDWKSINDQQGIDTHYLPGVNRGFAPGRLSHFFQWAGGFYSIDTGCSSSATALCLARDALTAGKYDAAVVGGGTLLTAPEWFAGLSQGGFLSPTGACKTYSDSADGYCRGEGVGVVILKRLADAVRSKDNVIAVIAGASRNCNAGAGSITYPGEKAQGALYRRVMRQAAVRPEQVDVVEMHGTGTQAGDRVETHAVQSVFAPSNGNQREKPLIVGALKANIGHSEAAAGIISLMKAILILQHDKIPAQPNQPIKMNPYLEPLIGKQIQLANGQSWTRNGAEPRYIFVNNFDAAGGNVSMLLQDPPAFALPAPASGPGLRTHHVVVTSGRTATAHEANRKRLHAYLSAHPDTNLADLAYTTTARRIHNVHREAYVASSTSDLVRQLEKPLADKVESAPPPAVVFTFTGQGAQSLGMGGALYSTSPTFRRLLDSLQSICEVQGLPTKFLNAIRGSGAEGATVTEVDMQVATVALEIALARYWRSLGIRPTVLIGHSLGEYAALCVAGVLSASDALALAFRRATLIFTRCPPSEAAMLAVGLPMRTVQYRIRDSAATTGCEVCCVNGPSSTVVGGPVAAIQALDEYLKSDGKVSTTRLRVQHAFHTRQMDVLLDELEASAAQVPFHAPTLPVASTVLGRIVRPGEQGVFDANYLRRHTREPVAFLDAVRACETEGLIPDRSFAVEIGPHPICISLMATCLQSAKINAWPSLRRGGDDWQSVSSTLAAAHSAQLPVAWSEFHKDHLDTVRLISDLPTYAFDLKTFWHSYKTPAAAVSAASATPSTTGLSRLASTTLHAVEKLQREEGKILGTFTVDLSDPKLAKAICGHVVDESAICPASIFIDMAYTAAVFLEQENGAGAALNTYELSSLEMHSPLVLREDIEVLPQVWVEAVLDIKSNAVSVHFKGQTSKGAVGYGSATMRLGQPDSAVRRDWSRIQSLVRARVQTLNRSVRPREVHAMDTALFYKVFSEIVDYSAPYHAVQEAVIAADFHDAAVTLQLTPTADLGTFTSSPFAVDALVHVAGFLLNADVRRPKNEVHIANHIGSLRIVGDLSSPGPYHVYATIREQDQKAGTSLCDVYTTDSQDRLVAVCSDICFKKLERDFFALLTGATRGRSTKPVAAAPAKSMAKRARQLAPSPSPSSSSGSNTPMSRSPTPSSVSDMVDLGTELLQAVAEQTGVSVAEMKSSPGTTFTEFGVDSQMAISILANFQRTTAVELPAAFFTNFPTPADAEAELGGSALDDLEEDITKPTPSPEQTQARKQGPAPSQHLLSLVAQALGLEASDLTPSTTFDSVGMDSMLSIKITAAFHAKTGIELPAAFFSANPTVGAAQEALDDDAEEESAPAQTSTNPAKETTIDSSRQHKLDAAVSRASYIHLKALPKGRRIYALESPFLEQPELFDLSIEEMATIFLRTIRRIQPHGPYLIGGWSAGSMYAYEVAHRLTREGETIQALIILDMRAPSLIPTSIVTTDFVDKLGTFEGINRARDLPEDLSVKERAHLMATCRALSRYDAPAFPSDRQPKQVAVVWALLGLDNRPDAPIASMGRPGLDIGKSMYEMNLDEFERYFNSWFYGRRQQFGTNGWEDLLGDHIAVYTVNGDHFSMMCPPYASEVGDIVIETVTRAVE

SEQ ID NO: 21 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 22 - protein, 5 aa, Cannabis sativa
YTPRK

(19) United States
(12) Patent Application Publication
(10) Pub. No.: US 2021/0403959 A1
(43) Pub. Date: Dec. 30, 2021

(54) USE OF TYPE I AND TYPE II POLYKETIDE SYNTHASES FOR THE PRODUCTION OF CANNABINOIDS AND CANNABINOID ANALOGS

(71) Applicant: BayMedica, Inc., Incline Village, NV (US)

(72) Inventors: Philip J. BARR, Oakland, CA (US); Charles K. MARLOWE, Emerald Hills, CA (US); Jianping SUN, Redwood City, CA (US); James T. Kealey, Sebastopol, CA (US)

(21) Appl. No.: 17/293,891

(22) PCT Filed: Nov. 13, 2019
(86) PCT No.: PCT/US2019/061289

§ 371 Date: May 13, 2021

Related U.S. Application Data
(60) Provisional application No. 62/767,428, filed on Nov. 14, 2018.

(51) Int. Cl. C12P 7/42; C12N 15/52; C12N 9/02; C12N 9/00; C12N 9/88; C12N 9/10

(52) U.S. Cl. CPC C12P 7/42; C12N 15/52; C12N 9/0071; C12Y 114/1201; C12Y 205/01102; C12N 9/93; C12N 9/88; C12Y 404/01026; C12N 9/1085; C12N 9/0095

(57) ABSTRACT

The present invention relates generally to production methods, enzymes and recombinant yeast strains for the biosynthesis of clinically important prenylated polyketides of the cannabinoid family. Using readily available starting materials, heterologous enzymes are used to direct cannabinoid biosynthesis in yeast.

### CROSS-REFERENCE TO RELATED APPLICATIONS

This application is a U.S. National Phase of International Application No. PCT/US2019/061289, filed Nov. 13, 2019, which claims priority benefit of U.S. provisional application No. 62/767,428, filed Nov. 14, 2018, each of which applications is herein incorporated by reference for all purposes.

### SEQUENCE LISTING

This application contains a Sequence Listing submitted electronically in ASCII format and is hereby incorporated by reference in its entirety. Said ASCII copy, created on May 12, 2021, is named 104059_1246789_SEQ_LST.txt and is 117,202 bytes in size.

### FIELD OF THE INVENTION

The present invention relates generally to production methods, enzymes and recombinant yeast strains for the biosynthesis of clinically important polyketides of the cannabinoid family. Using readily available starting materials, heterologous enzymes are used to direct cannabinoid and cannabinoid analog biosynthesis in eukaryotic microorganisms, e.g., yeast.

### BACKGROUND OF THE INVENTION

Cannabis sativa varieties have been cultivated and utilized extensively throughout the world for a number of applications. Currently, cannabinoids are isolated primarily via the cultivation of large acreages of cannabis or hemp plants in agricultural operations throughout the world, with a lower, albeit clinically important level of production methodologies that involve synthetic chemical processes.

Synthetic biology, whereby individual cannabinoids are biosynthesized using isolated genetic pathways in engineered microorganisms, allows for commercial manufacture and large scale production of naturally occurring cannabinoids and their analogs as highly pure compounds with full biological and pharmacological activities.

In C. sativa, the first chemical building blocks of the cannabinoid molecules and their analogs are polyketides. Polyketides generally are synthesized by condensation of two-carbon units in a manner analogous to fatty acid synthesis. In general, the synthesis involves a starter unit and extender units; these starter units are derived from, for example, acylthioesters, typically acetyl-, coumaroyl-, propionyl-, malonyl- or methylmalonyl-coenzyme-A (CoA) thioesters. The first enzymatic step in the biosynthesis of the more prevalent cannabinoids in C. sativa, however, is the formation of olivetolic acid by a type III polyketide synthase (PKS) enzyme that catalyzes the condensation of hexanoyl-CoA with three molecules of malonyl-CoA to form a tetraketide that is then cyclized and aromatized by a separate gene-encoded cyclase enzyme. The major cannabinoids, including 49-tetrahydrocannabinolic acid and cannabidiolic acid, are thus formed from the initiating precursor hexanoyl-CoA, a medium chain fatty acyl-CoA. Other, less prevalent cannabinoids with variant side-chains are formed from aliphatic-CoAs of different lengths (e.g. 49-tetrahydrocannabivarinic acid is formed from an n-butanoyl-CoA starter unit). Several additional and related analogs are found in nature, and others have been chemically synthesized.

PKSs are analogous to fatty acid synthases. The greater structural diversity of polyketide products stems from the fact that PKSs can vary the degree of reduction after each step. This can lead to formation of a ketone, hydroxyl, alkene or methylene functionality at C-3 in the chain after each condensation. Additional diversity arises because PKSs do not only use malonyl-CoA as an extender unit. Systems that use methylmalonyl-CoA and methoxymalonyl-CoA are also known. PKSs can utilize a wide variety of starter units and also feature C-methylation domains for the introduction of branching. Type I modular PKSs are analogous to Type I FASs in that all the domains are present on a single polypeptide. Unlike FAS, however, each domain is only used once. The domains are formed into modules which collectively perform one condensation step and associated modification of the polyketide chain before transfer to the following module.

The first known modular PKS was 6-deoxyerythronolide B synthase (DEBS) from Saccharopolyspora erythraea. Sequence analysis of the S. erythraea genome found three large open reading frames (ORFs) which encoded three very large polypeptides (approximately 350 kDa each). By sequence comparison to FAS domains, regions of the polypeptides were assigned biosynthetic functions. The DEBS megasynthases function as a ‘molecular assembly line’, passing the growing polyketide chain from one module. The sequence of domains corresponds exactly to the functionality observed in the product 6-deoxerythronolide B (6-dEB) Not all Type I modular PKSs conform to this rule. The rapamycin PKS, for example, contains modules that have KR, DH and ER domains that are not required to act to form the final product. Modular Type I PKSs are dimeric and have been proposed to adopt the same structure as mFAS, a head-to-head, tail-to-tail dimer. This structure is more complicated than the iterative mFAS since a modular PKS can contain more than one covalently linked set of modules and must also be able to interact with modules on other polypeptide chains.

Type I iterative PKSs are mostly found in fungi and consist of a single large polypeptide with multiple domains distributed along it. Fungal PKSs use a single set of active sites iteratively, and can be subdivided into three classes based on their product: highly-reducing, partially reducing and non-reducing. Highly-reducing fungal PKSs, such as the lovastatin synthases LovB and LovF, yield products with a high degree of saturation. Partially-reducing PKSs are typified by 6-methylsalcylic acid synthase (6-MSAS). This performs only one ketoreduction in three condensation cycles to form the aromatic compound 6-MSA. The non-reducing PKSs form aromatic compounds such as orsellinic acid, olivetolic and divarinic acids, with the latter two being starter units for prenylation (geranylation) to form cannabinoid precursors and their analogs.

Although all three classes of type I iterative PKSs carry out similar reactions, the makeup of their synthases are very different. Highly reducing PKSs feature ketosynthase (KS), acyltransferase (AT), ketoreductase (KR), dehydratase (DH), enoylreductase (ER) and acyl carrier protein (ACP) domains, along with a C-methyltransferase domain. Non reducing-PKSs lack any domains from the reductive loop, but instead contain starter unit:acyl-carrier protein transacylase (SAT) and product template (PT) domains, alongside Claisen cyclase domains or thioesterase (TE) domains for off-loading. Partially reducing PKSs have a simple domain structure, containing only KS, AT, DH, KR and ACP domains along with a core domain of unknown function.

The SAT domain is responsible for the selection of the initial acid CoA derivative that, in many PKSs is acetyl-CoA, but in the natural biosynthesis of cannabinoids in C. sativa is hexanoyl- or butanoyl-CoA.

Type II PKSs, like bacterial type II FASs, are associated complexes of discrete proteins. The “minimal PKS” consists of two KS-like enzymes (KSα and KSβ). KSβ has [symbol] been shown to be important in controlling chain length of products and is also known as the ‘chain length factor’ (CLF). Other proteins encoding ketoreductases, aromatases and cyclases can also act on the polyketide chain.

Type III PKSs, like type II PKSs act in an iterative manner. Instead of the multi-enzyme complex, a single KS-like domain is used to carry out all decarboxylation, condensation, cyclisation and aromatisation reactions. Rather than utilising substrates bound to an ACP, type III PKSs act on CoA thioesters directly. Type III PKSs such as olivetolic acid synthase, resveratrol synthase and chalcone synthase use a wide variety of acyl-CoA starter units to generate diversity and typically give mono- and bi-cyclic aromatic products.

### BRIEF SUMMARY OF ASPECTS OF THE INVENTION

This summary highlights only certain aspects of the disclosure and does not include a description of all aspects of the invention.

In one aspect, the present disclosure describes the use of modified iterative Type I PKSs or Type II PKSs that have been repurposed to catalyze the assembly of the polyketide precursors of cannabinoids. Use of a Type I PKS or Type II PKS can provide a more rapid rate of synthesis and generate higher levels of cannabinoid precursors.

In one aspect, provided herein is a modified recombinant host cell comprising: (i) a first exogenous polynucleotide that encodes a BenA polypeptide comprising an amino acid sequence having at least 90% or at least 95% identity to SEQ ID NO:16 (ii) a second exogenous polynucleotide that encodes a BenB polypeptide comprising an amino acid sequence having at least 90% or least 95% identity to SEQ ID NO:17, (iii) a third exogenous polynucleotide that encodes a BenC polypeptide comprising an amino acid sequence having at least 90% or at least 95% amino acid identity to SEQ ID NO:18. In some embodiments, the modified recombinant host cell further comprises an exogenous polynucleotide a 2-alkyl-4,6-dihydroxybenzoic acid cyclase. In some embodiments, the 2-alkyl-4,6-dihydroxybenzoic acid cyclase is a truncated olivetolic acid cyclase, an AtHS1 polypeptide, or the N-terminal domain of a BenH polypeptide. In some embodiments, the modified host cell comprises a fourth exogenous polynucleotide that encodes a BenH polypeptide comprising an amino acid sequence having at least 90% or at least 95% identity to SEQ ID NO:13. In some embodiments, the BenH polypeptide comprises an amino acid sequence having at least 90% or at least 95% identity to SEQ ID NO:19. In some embodiments, the modified recombinant host cell comprises (i) a first exogenous polynucleotide that encodes a BenA polypeptide comprising the amino acid sequence of SEQ ID NO:16 (ii) a second exogenous polynucleotide that encodes a BenB polypeptide comprising the amino acid sequence of SEQ ID NO:17, and (iii) a third exogenous polynucleotide that encodes a BenC polypeptide comprising the amino acid sequence of SEQ ID NO:18. In some embodiments, the modified recombinant host cell comprises a fourth exogenous polynucleotide encoding a BenH polypeptide comprising the amino acid sequence of SEQ ID NO:19. In some embodiments, a modified recombinant host cell as described herein, e.g., in this paragraph, comprises an exogenous polynucleotide encoding an olivetolic acid synthase (also known as a tetraketide synthase) polypeptide from C. sativa. In some embodiments, the olivetolic acid synthase polypeptide comprises an an amino acid sequence having at least 90% or at least 95% identity to SEQ ID NO:21. In some embodiments, the olivetolic acid synthase polypeptide comprises the amino acid sequence SEQ ID NO:21. In some embodiments, the modified recombinant host cell comprises an exogenous polynucleotide encoding an olivetolic acid synthase from C. sativa and an exogenous polynucleotide encoding a BenH polypeptide, e.g., a BenH polypeptide comprising an amino acid sequence having at least 90% or at least 95% identity to SEQ ID NO:13. In some embodiments, the BenH polypeptide comprises SEQ ID NO:13. In some embodiments, the modified recombinant host cell is a yeast cell genetically modified to knockout expression of the PAD1 and FDC1 aromatic decarboxylase genes. In some embodiments, one or more of the exogenous polynucleotides is present in an autonomously replicating expression vector. For example, in some embodiments, the exogenous polynucleotide encoding the BenA, BenB, and BenC are contained in the same autonomously replicating expression vector and expressed as a multicistronic mRNA. In some embodiments, the autonomously replicating expression vector is a yeast artificial chromosome. In other embodiments, one or more of the exogenous polynucleotides are integrated into the host genome. Such exogenous polynucleotide may, for example, be introduced into the recombinant host cell by retrotransposon integration. In some embodiments, expression of one or more of the exogenous polynucleotides is driven by an alcohol dehydrogenase-2 promoter. In some embodiments, the host cell is a cell selected from the group consisting of a Saccharomyces cerevisiae, Kluyveromyces lactis, Kluyveromyces marxianus, Pichia pastoris, Yarrowia lipolytica, Hansenula polymorpha and an Aspergillus cell.

In one aspect, provided herein is a method of producing a cannabinoid product or a cannabinoid precursor product, the method comprising culturing a modified recombinant host cell of the preceding paragraph under conditions in which the exogenous polynucleotides are expresses thereby producing the cannabinoid product or cannabinoid precursor product.

In a further aspect, provided herein is a method of producing a cannabinoid product, the method comprising culturing a modified recombinant host cell comprising: (i) a first exogenous polynucleotide that encodes a BenA polypeptide; (ii) a second exogenous polynucleotide that encodes a BenB polypeptide; (iii) a third exogenous polynucleotide that encodes a BenC polypeptide; and optinally, a fourth exogenous polynucleotide that encodes the N-terminal domain of a BenH polypeptide; under conditions in which products encoded by the exogenous polynucleotides are expressed and a 5-alkyl-benzene-1,3-diol is produced; and converting the 5-alkyl-benzene-1,3-diol to the cannabinoid product. In some embodiments, the 5-alkyl-benzene-1,3-diol is olivetol. In some embodiments, the converting step comprises forming a reaction mixture comprising the olivetol, citral, and an amine and maintaining the reaction mixture under conditions sufficient to produce cannabichromene (CBC).

In one aspect, provided herein are genetically modified recombinant host cells for cannabinoid expression that employ a Type I or Type II PKS for cannabinoid expression. The host cells are modified to express an exogenous polynucleotide that encodes a Type I PKS, e.g., a micacocdin PKS, or a Type II PKS, e.g. benastatin. The cells additionally comprise an exogenous polynucleotide that encodes an acyl-CoA synthetase that converts an aliphatic carboxylic acid to an acyl CoA thioester, e.g., a RevS polypeptide or a CsAAE3 polypeptide. In some embodiments, the recombinant host cells comprise an exogenous polynucleotide that encodes a cyclase, e.g., a truncated olivetolic acid cyclase or an olivetolic acid cyclase homolog, such as AtHS1, or the amino-terminal domain of the BenH protein, from a benastatin-producing gene cluster, e.g., from Streptomyces sp. A2991200.

Thus, in in one aspect, provided herein is a modified recombinant host cell comprising: (i) a first exogenous polynucleotide that encodes an acyl-CoA synthetase that converts an aliphatic carboxylic acid to an acyl CoA thioester, (ii) a second exogenous polynucleotide that encodes a Type I polyketide synthase (PKS), (iii) and a third exogenous polynucleotide that encodes a 2-alkyl-4,6-dihydroxybenzoic acid cyclase. In some embodiments, the aliphatic carboxylic acid is hexanoic or butanoic acid. In some embodiments the Type I PKS is a MicC PKS. In further embodiments, the modified recombinant host cell comprises an exogenous polynucleotide that encodes a phosphopantotheinyl transferase (PPTas). In some embodiments, the PPTase is a MicA polypeptide. Alternatively, the PPTase may be a phosphopantetheinyl transferase from Aspergillus, e.g., NpgA or PptB or a bacterial phosphopantetheinyl transferase, such as sfp, e.g., from Bacillus. In further embodiments, the 2-alkyl-4,6-dihydroxybenzoic acid cyclase is olivetolic acid cyclase, e.g., a truncated olivetolic acid cyclase from C. sativa, or the AtHS1 or the amino-terminal domain of the BenH protein from a benastatin gene cluster, e.g., from Streptomyces sp. A2991200.

In an additional aspect, provided herein is a modified recombinant host cell comprising: (i) a first exogenous polynucleotide that encodes an acyl-CoA synthetase that converts an aliphatic carboxylic acid to an acyl CoA thioester, and (ii) a second exogenous polynucleotide that encodes a MicC PKS that comprises a mutation in a ketoreductase (KR) domain that inactivates the KR domain, such that the MicC PKS produces a 2-alkyl-4,6-dihydroxybenzoic acid from the acyl-CoA. In some embodiments, the aliphatic carboxylic acid is hexanoic acid or butanoic acid. In some embodiments, the modified recombinant host cell further comprises an exogenous polynucleotide that encodes a PPTase, for example, a PPTase such as a MicA polypeptide, or a NpgA (Uniprotein G5EB87) or sfp (Uniprotein P39135) polypeptide. In further embodiments, the acyl-CoA synthetase is a revS polypeptide; or a transmembrane domain-deleted CsAAE1 or a CsAAE3 from C. sativa.

In a further aspect, provided herein is a modified recombinant host cell comprising: (i) a first exogenous polynucleotide that encodes an acyl-CoA synthetase that converts an aliphatic carboxylic acid to an acyl CoA thioester, (ii) a second exogenous polynucleotide that encodes a Type II polyketide synthase (PKS), (iii) and a third exogenous polynucleotide that encodes a 2-alkyl-4,6-dihydroxybenzoic acid cyclase. In some embodiments, the aliphatic carboxylic acid is hexanoic acid or butanoic acid. In some embodiments, the Type II PKS is a BenA PKS, or a mulitmeric BenA-BenB-BenC PKS. In some embodiments, the modified recombinant host cell further comprises an exogenous polynucleotide encoding a BenQ polypeptide. In some embodiments, the 2-alkyl-4,6-dihydroxybenzoic acid cyclase is olivetolic acid cyclase, e.g., a truncated olivetolic acid cyclase. In some embodiments, the acyl-CoA synthetase is a revS polypeptide; or a transmembrane domain-deleted CsAAE1 or a CsAAE3 from C. sativa.

In some embodiments, the aliphatic carboxylic acid is selected from hexanoic or butanoic acid, such that the resulting cannabinoid or cannabinoid precursor contain the natural pentyl- or propyl-substituted aromatic ring,

In some embodiments, the carboxylic acid may contain 2-12 linear or branched carbon atoms and may contain C—C double bonds.

In some embodiments, the carboxylic acid may contain 2-12 linear or branched carbon atoms and may contain C—C double bonds wherein hydrogen atoms are substituted as described hereinbelow.

In some embodiments, the disclosure provides a modified recombinant host cell as described herein, e.g., in the preceding three paragraphs, where the modified host cell further comprises an exogenous polynucleotide that encodes a prenyltransferase that catalyzes coupling of geranyl-pyrophsophate to a 2-alkyl-4,6-dihydroxybenzoic acid to produce an acidic cannabinoid.

In some embodiments, the disclosure provides a modified recombinant host cell as described herein, e.g., in the preceding paragraphs in the section, wherein the modified recombinant host cell is a yeast cell genetically modified to knockout expression of the PAD1 and FDC1 aromatic decarboxylase genes.

In some embodiments one or more of the exogenous polynucleotides as described herein, e.g., in the preceding paragraphs in this section, is present in an autonomously replicating expression vector, such as a plasmid or a yeast artificial chromosome.

In some embodiments, a modified recombinant host cell as described herein comprises an exogenous polynucleotide encoding MicC and an exogenous polynucleotide encoding MicA contained in the same autonomously replicating vector. In some embodiments, the MicC and MicA mRNAs are expressed as components of a multicistronic mRNA.

In some embodiments, a modified recombinant host cell as described herein comprises an exogenous polynucleotide encoding BenA and an exogenous polynucleotide encoding BenQ contained in the same autonomously replicating vector. In some embodiments, the BenA and BenQ mRNAs are expressed as components of a multicistronic mRNA.

In some embodiments one or more of the exogenous polynucleotides as described herein, e.g., in the preceding paragraphs, is integrated into the host genome. In some embodiments, the one or more exogenous polynucleotides are introduced into the recombinant host cell by retrotransposon integration.

In some embodiments, expression of one or more of the exogenous polynucleotides in a modified recombinant host cell as described herein, e.g., the preceding paragraphs is driven by an alcohol dehydrogenase-2 promoter.

In some embodiments, the modified recombinant host cell as described herein is a cell selected from the group consisting of a Saccharomyces cerevisiae, Kluyveromyces lactis, Kluyveromyces marxianus, Pichia pastoris, Yarrowia lipolytica, Hansenula polymorpha and Aspergillus cell.

In a further aspect, provided herein is a method of producing a cannabinoid product, the method comprising culturing a modified recombinant host cell as described herein, e.g., in the preceding paragraphs, under conditions in which the exogenous polynucleotides are expressed thereby producing the cannabinoid product.

The disclosure further provides a method of producing a cannabinoid product, the method comprising culturing a modified recombinant host cell comprising: (i) a first exogenous polynucleotide that encodes an acyl-CoA synthetase that converts an aliphatic carboxylic acid to an acyl CoA thioester; (ii) a second exogenous polynucleotide that encodes a Type I polyketide synthase (PKS) that produces a polyketide from the acyl CoA thioester and malonyl CoA; (iii) a third exogenous polynucleotide that encodes a 2-alkyl-4,6-dihydroxybenzoic acid cyclase; under conditions in which products encoded by the exogenous polynucleotides are expressed and a 2-alkyl-4,6-dihydroxybenzoic acid is produced; and converting the 2-alkyl-4,6-dihydroxybenzoic acid to the cannabinoid product. In some embodiments, the aliphatic carboxylic acid is hexanoic acid. In some embodiments, the Type I PKS is a MicC PKS. In some embodiments, the modified recombinant host cell further comprises an exogenous polynucleotide that encodes a PPTase for example, a MicA PPTase. In some embodiments, the 2-alkyl-4,6-dihydroxybenzoic acid cyclase is olivetolic acid cyclase, e.g., a truncated olivetolic acid cyclase, or is AtHS1, or the amino-terminal domain of a BenH protein from a benastatin gener cluster, e.g., from Streptomyces sp. A2991200. In some embodiments, the acyl-CoA synthetase is a revS polypeptide; or a transmembrane-deleted CsAAE1 or a CsAAE3 polypeptide from C. sativa.

In a further aspect, provided herein is a method of producing a cannabinoid product, the method comprising culturing a modified recombinant host cell comprising: (i) a first exogenous polynucleotide that encodes an acyl-CoA synthetase that converts an aliphatic carboxylic acid to an acyl CoA thioester; and (ii) a second exogenous polynucleotide that encodes a MicC polypeptide that comprises a mutation in a ketoreductase (KR) domain that inactivates the KR domain to produce a 2-alkyl-4,6-dihydroxybenzoic acid from the acyl CoA thioester and malonyl CoA. In some embodiments, the aliphatic carboxylic acid is hexanoic or butanoic acid. In some embodiments, the host cell is genetically modified to comprise an exogenous polynucleotide encoding a PPTase, e.g., a MicA polypeptide. In some embodiments, the 2-alkyl-4,6-dihydroxybenzoic acid is olivetolic acid. In some embodiments, the acyl-CoA synthetase is a revS polypeptide; or is a transmembrane-deleted CsAAE1 polypeptide or a CsAAE3 polypeptide from C. sativa. In some embodiments, the 2-alkyl-4,6-dihydroxybenzoic acid cyclase comprises a DABB domain. In further embodiments, the modified recombinant host cell is a yeast cell genetically modified to knockout expression of the PAD1 and FDC1 aromatic decarboxylase genes.

The disclosure additionally provides a method of producing a cannabinoid product, the method comprising culturing a modified recombinant host cell comprising: (i) a first exogenous polynucleotide that encodes an acyl-CoA synthetase that converts an aliphatic carboxylic acid to an acyl-CoA thioester, (ii) a second exogenous polynucleotide that encodes a Type II polyketide synthase (PKS), (iii) and a third exogenous polynucleotide that encodes a 2-alkyl-4,6-dihydroxybenzoic acid cyclase. In some embodiments, the aliphatic carboxylic acid is hexanoic acid. In some embodiments, the Type II PKS is a BenA PKS. In additional embodiments, the modified recombinant host cell further comprises an exogenous polynucleotide encoding a BenQ polypeptide. In some embodiments, the 2-alkyl-4,6-dihydroxybenzoic acid cyclase is olivetolic acid cyclase, e.g., a truncated olivetolic acid cyclase. In some embodiments, the acyl-CoA synthetase is a revS polypeptide; or a transmembrane-deleted CsAAE1 polypeptide or a CsAAE3 polypeptide from C. sativa.

In some embodiments of a method as disclosed herein, e.g., in the preceding paragraphs, the modified recombinant host cell further comprises an exogenous polynucleotide that encodes a prenyltransferase that catalyzes coupling of geranyl-pyrophsophate to a 2-alkyl-4,6-dihydroxybenzoic acid to produce an acidic cannabinoid. In some embodiments of a method as disclosed herein, the modified recombinant host cell is a yeast cell genetically modified to knockout expression of the PAD1 and FDC1 aromatic decarboxylase genes.

In some embodiments, the 2-alkyl-4,6-dihydroxybenzoic acid is the cannabinoid product. In further embodiments, the method further comprises converting the 2-alkyl-4,6-dihydroxybenzoic acid to the cannabinoid product.

In some embodiments, the 2-alkyl-4,6-dihydroxybenzoic acid is converted to the cannabinoid product in vitro. In some embodiments, the 2-alkyl-4,6-dihydroxybenzoic acid is olivetolic acid and the converting step comprises forming a reaction mixture comprising the olivetolic acid, geraniol, and an organic solvent and maintaining the reaction mixture under conditions sufficient to produce a cannabigerolic acid (CBGA). In some embodiments, the reaction mixture further comprises an acid, e.g., p-toluenesulfonic acid. In some embodiments the organic solvent is toluene. In further embodiments, the reaction mixture comprises the host cell.

Also provided herein are methods for producing cannabinoid products comprising culturing a modified recombinant host cell comprising (i) a first exogenous polynucleotide that encodes an acyl-CoA synthetase that converts an aliphatic carboxylic acid to an acyl CoA thioester; (ii) a second exogenous polynucleotide that encodes a Type I PKS or a Type III PKS that that produces a tetraketide from an Acyl-CoA and malonyl CoA; (iii) and optionally, a third exogenous polynucleotide that encodes a cyclase, e.g., olivetolic acid cyclase; under conditions in which products encoded by the exogenous polynucleotides are expressed and olivetolic acid is produced; and converting the olivetolic acid to the cannabinoid. The conversion can be conducted chemically or enzymatically, in vitro or in vivo.

In some embodiments, an acyl CoA thioester is generated by chemical synthesis rather than enzymatically using an acyl-CoA synthetase. Accordingly, in some embodiments, a genetically modified host cell that expresses an exogenous Type I or Type II PKS need not be engineered to express an exogenous acyl-CoA synthetase.

### BRIEF DESCRIPTION OF THE DRAWINGS

FIG. 1 depicts a synthesis scheme to generate cannabinoids.

FIG. 2 provides illustrative data showing production of olivetol and olivetolic acid in a yeast strain expressing BenA, BenB and BenC genes on one plasmid, and benH on a second plasmid (left), compared with a control expressing the Cs tetraketide synthase and benH (right).

### DETAILED DESCRIPTION OF THE INVENTION

### I. Introduction

The present invention provides methods and materials for producing cannabinoid compounds of interest in a rapid, inexpensive and efficient manner using Type I or Type II PKSs.

In one aspect, the present invention provides novel systems for the efficient production of the prenylated polyketides (Page, J. E., and Nagel, J. (2006). Biosynthesis of terpenophenolics in hop and cannabis. In Integrative Plant Biochemistry, J. T. Romeo, ed, (Oxford, UK: Elsevier), pp. 179-210), that comprise the cannabinoid family along with cannabinoid precursor molecules and their analogs, using commercial yeast biopharmaceutical manufacturing systems. In some embodiments, the yeast strains chosen as hosts belong to the Saccharomyces cerevisiae species of yeast that does not produce such molecules naturally. Other species of yeasts that may be employed include, but are not limited to, Kluyveromyces lactis, K. marxianus, Pichia pastoris, Yarrowia lipolytica, and Hansenula polymorpha. Similarly, certain Aspergillus species may also be engineered for cannabinoid production.

The present invention can employ coding sequences from both type I PKSs and type II PKSs. Genes encoding polypeptide components of type I PKSs have been used for the microbiological production of similar polyketides in heterologous microorganisms such as yeast and E. coli. See for example U.S. Pat. Nos. 6,033,883, 6,258,566, 7,078,233 and 9,637,763 and Kealey et al., Proc Natl Acad Sci USA (1998) 95, 505

### II. Definitions

Unless otherwise defined, all terms of art, notations and other scientific terminology used herein are intended to have the meanings commonly understood by those of ordinary skill in the art to which the present application pertains. In some cases, terms with commonly understood meanings are defined herein for clarity and/or for ready reference, and the inclusion of such definitions herein should not necessarily be construed to represent a substantial difference over what is generally understood in the art.

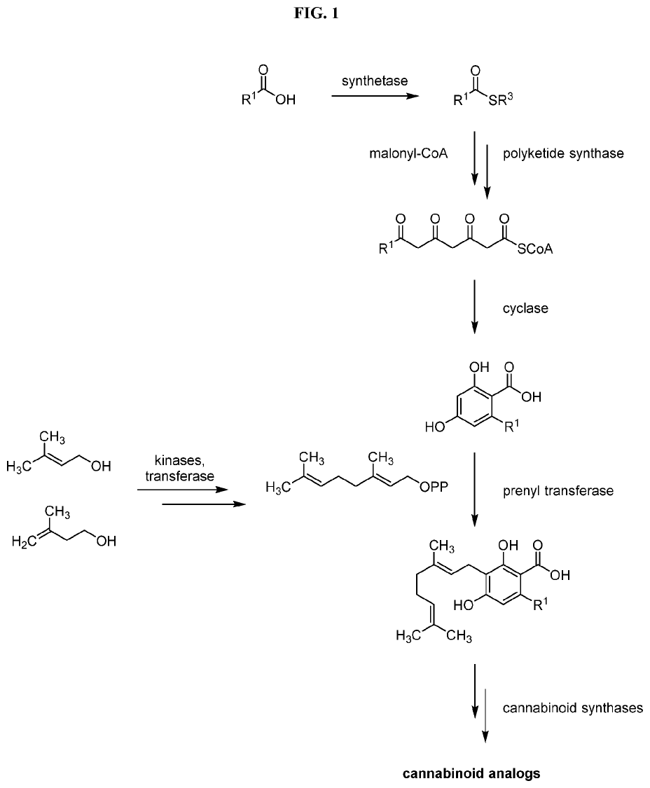

As used herein, the terms “cannabinoid,” “cannabinoid compound,” and “cannabinoid product” are used interchangeably to refer to a molecule containing a polyketide moiety, e.g., olivetolic acid or another 2-alkyl-4,6-dihydroxybenzoic acid, and a terpene-derived moiety e.g., a geranyl group. Geranyl groups are derived from the diphosphate of geraniol, known as geranyl pyrophosphate, which can react with olivetolic acid type compounds to form the acidic cannabinoid cannabigerolic acid (CBGA) and CBGA analogs, as shown in FIG. 1. CBGA can be converted to further bioactive cannabinoids both enzymatically (e.g., by decarboxylation via enzyme treatment in vivo or in vitro) and chemically (e.g. by heating).

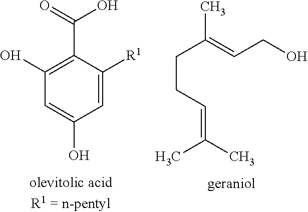

The term cannabinoid includes acid cannabinoids and neutral cannabinoids. The term “acidic cannabinoid” refers to a cannabinoid having a carboxylic acid moiety. The carboxylic acid moiety may be present in protonated form (i.e., as —COOH) or in deprotonated form (i.e., as carboxylate —COO—). Examples of acidic cannabinoids include, but are not limited to, cannabigerolic acid, cannabidiolic acid, cannabichromenic acid and Δ9-tetrahydrocannabinolic acid. The term “neutral cannabinoid” refers to a cannabinoid that does not contain a carboxylic acid moiety (i.e., does not contain a moiety —COOH or —COO—). Examples of neutral cannabinoids include, but are not limited to, cannabigerol, cannabidiol, cannabichromene and Δ9-tetrahydrocannabinol.

The term “2-alkyl-4,6-dihydroxybenzoic acid” refers to a compound having the structure:

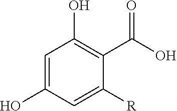

wherein R is a C₁-C₂₀ alkyl group, which in some embodiments, can be halogenated, hydroxylated, deuterated, and/or tritiated. Examples of 2-alkyl-4,6-dihydroxybenzoic acids include, but are not limited to olivetolic acid (i.e., 2-pentyl-4,6-dihydroxybenzoic acid; CAS Registry No. 491-72-5) and divarinic acid (i.e., 2-propyl-4,6-dihydroxybenzoic acid; CAS Registry No. 4707-50-0). Olivetolic acid analogs include other 2-alkyl-4,6-dihydroxybenzoic acids and substituted resorcinols including, but not limited to, 5-halomethylresorcinols, 5-haloethylresorcinols, 5-halopropylresorcinols, 5-halohexylresorcinols, 5-haloheptylresorcinols, 5-halooctylresorcinols, and 5-halononylresorcinols.

The term “prenyl moiety” refers to a substituent containing at least one methylbutenyl group (e.g., a 2-methylbut-2-ene-1-yl group). In many instances prenyl moieties are synthesized biochemically from isopentenyl pyrophosphate and/or isopentenyl diphosphate giving rise to terpene natural products and other compounds. Examples of prenyl moieties include, but are not limited to, prenyl, geranyl, myrcenyl, ocimenyl, farnesyl, and geranylgeranyl.

The term “geraniol” refers to (2E)-3,7-dimethyl-2,6-octadien-1-ol (CAS Registry No. 106-24-1). The term “geranylating” refers to the covalent bonding of a 3,7-dimethyl-2,6-octadien-1-yl radical to a molecule such as a 2-alkyl-4,6-hydroxybenzoic acid. Geranylation can be conducted chemically or enzymatically, as described herein.

The term “2-alkyl-4,6-dihydroxybenzoic acid” refers to a compound having the structure:

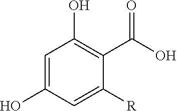

wherein R is a C₁-C₂₀ alkyl group. Examples of 2-alkyl-4,6-dihydroxybenzoic acids include, but are not limited to olivetolic acid (i.e., 2-pentyl-4,6-dihydroxybenzoic acid; CAS Registry No. 491-72-5) and divarinic acid (i.e., 2-propyl-4,6-dihydroxybenzoic acid; CAS Registry No. 4707-50-0). Olivetolic acid analogs include other 2-alkyl-4,6-dihydroxybenzoic acids and substituted resorcinols such as 5-methylresorcinol, 5-ethylresorcinol, 5-propylresorcinol, 5-hexylresorcinol, 5-heptylresorcinol, 5-octylresorcinol, and 5-nonylresorcinol.

The term “alkyl,” by itself or as part of another substituent, refers to a straight or branched, saturated, aliphatic radical. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

The term “alkenyl,” by itself or as part of another substituent, refers to an alkyl group, as defined herein, having one or more carbon-carbon double bonds. Examples of alkenyl groups include, but are not limited to, vinyl (i.e., ethenyl), crotyl (i.e., but-2-en-1-yl), penta-1,3-dien-1-yl, and the like. Alkenyl moieties may be further substituted, e.g., with aryl substituents (such as phenyl or hydroxyphenyl, in the case of 4-hydroxystyryl).

The terms “halogen” and “halo,” by themselves or as part of another substituent, refer to a fluorine, chlorine, bromine, or iodine atom.

The term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

The term “hydroxyalkyl,” by itself or as part of another substituent, refers to an alkyl group where some or all of the hydrogen atoms are replaced with hydroxyl groups (i.e., —OH groups). As for alkyl and haloalkyl groups, hydroxyalkyl groups can have any suitable number of carbon atoms, such as C₁₋₆.

The term “deuterated” refers to a substituent (e.g., an alkyl group) having one or more deuterium atoms (i.e., ²H atoms) in place of one or more hydrogen atoms.

The term “tritiated” refers to a substituent (e.g., an alkyl group) having one or more ritium atoms (i.e., ³H atoms) in place of one or more hydrogen atoms.

An “organic solvent” refers to a carbon-containing substance that is liquid at ambient temperature and pressure and is substantially free of water. Examples of organic solvents include, but are not limited to, toluene, methylene chloride, ethyl acetate, acetonitrile, tetrahydrofuran, benzene, chloroform, diethyl ether, dimethyl formamide, dimethyl sulfoxide, and petroleum ether.

The term “acid” refers to a substance that is capable of donating a proton (i.e., a hydrogen cation) to form a conjugate base of the acid. Examples of acids include, but are not limited to, mineral acids (e.g., hydrochloric acid, sulfuric acid, and the like), carboxylic acids (e.g., acetic acid, formic acid, and the like), and sulfonic acids (e.g., methanesulfonic acid, p-toluenesulfonic acid, and the like).

Throughout this specification and claims, the word “comprise,” or variations such as “comprises” or “comprising,” will be understood to imply the inclusion of a stated integer or group of integers but not the exclusion of any other integer or group of integers.

The terms “identical” or percent “identity,” in the context of two or more polypeptide sequences, refer to two or more sequences or subsequences that are the same or have a specified percentage of amino acid residues that are the same (e.g., at least 70%, at least 75%, at least 80%, 85%, 90%, 91%, 92%, 93%, 94%, 95%, 96%, 97%, 98%, 99%, or higher) identity over a specified region, when compared and aligned for maximum correspondence over a comparison window or designated region. Alignment for purposes of determining percent amino acid sequence identity can be performed in various methods, including those using publicly available computer software such as BLAST, BLAST-2, ALIGN, Geneious, or Megalign (DNASTAR) software, among others. Examples of algorithms that are suitable for determining percent sequence identity and sequence similarity the BLAST 2.0 algorithms, which are described in Altschul et al., Nuc. Acids Res. 25:3389-3402 (1977) and Altschul et al., J. Mol. Biol. 215:403-410 (1990). Thus, BLAST 2.0 can be used with the default parameters described to determine percent sequence identity.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) aliphatic amino acids Gly, Ala, Val, Leu and Ile; (vi) slightly polar amino acids Met and Cys; (vii) small-side chain amino acids Ser, Thr, Asp, Asn, Gly, Ala, Glu, Gln and Pro; (viii) small hydroxyl amino acids Ser and Thr; and sulfur-containing amino acids Cys and Met. Reference to the charge of an amino acid in this paragraph refers to the charge at pH 7.0.

In specific cases, abbreviated terms are used. For example, the term “CBGA” refers to cannabigerolic acid. Likewise: “OA” refers to olivetolic acid; “CBG” refers to cannabigerol; “CBDA” refers to cannabidiolic acid; “CBD” refers to cannabidiol; “THC” refers to Δ⁹-tetrahydrocannabinol (Δ⁹-THC); “Δ⁸-THC” refers to Δ⁸-tetrahydrocannabinol; “THCA” refers to Δ⁹-tetrahydrocannabinolic acid (Δ⁹-THCA); “Δ⁸-THCA” refers to Δ⁸-tetrahydrocannabinolic acid; “CBCA” refers to cannabichromenic acid; “CBC” refers to cannabichromene; “CBN” refers to cannabinol; “CBND” refers to cannabinodiol; “CBNA” refers to cannabinolic acid; “CBV” refers to cannabivarin; “CBVA” refers to cannabivarinic acid; “THCV” refers to Δ⁹-tetrahydrocannabivarin (Δ⁹-THCV); “Δ⁸-THCV” refers to “Δ⁸-tetrahydrocannabivarin; “THCVA” refers to Δ⁹-tetrahydrocannabivarinic acid (Δ⁹-THCV); “Δ⁸-THCVA” refers to Δ⁸-tetrahydrocannabivarinic acid; “CBGV” refers to cannabigerovarin; “CBGVA” refers to cannabigerovarinic acid; “CBCV” refers to cannabichromevarin; “CBCVA” refers to cannabichromevarinic acid; “CBDV” refers to cannabidivarin; “CBDVA” refers to cannabidivarinic acid; “MPF” refers to multiple precursor feeding; “PKS” refers to a polyketide synthase; “GOT” refers to geranyl pyrophosphate olivetolate geranyl transferase; “YAC” refers to yeast artificial chromosome; “IRES” or “internal ribosome entry site” means a specialized sequence that directly promotes ribosome binding and mRNA translation, independent of a cap structure; and “HPLC” refers to high performance liquid chromatography.

As used herein and in the appended claims, the singular forms “a,” “and,” and “the” include plural referents unless the context clearly dictates otherwise.

As used herein, the terms “about” and “around” indicate a close range around a numerical value when used to modify that specific value. If “X” were the value, for example, “about X” or “around X” would indicate a value from 0.9X to 1.1X, e.g., a value from 0.95X to 1.05X, or a value from 0.98X to 1.02X, or a value from 0.99X to 1.01X. Any reference to “about X” or “around X” specifically indicates at least the values X, 0.9 X, 0.91X, 0.92X, 0.93X, 0.94X, 0.95X, 0.96X, 0.97×0.98×0.99X, 1.01×1.02×1.03X, 1.04, X 1.05X, 1.06X, 1.07X, 1.08X, 1.09X, and 1.1X, and values within this range

The techniques and procedures described or referenced herein are generally well understood and commonly employed using conventional methodology by those skilled in the art, such as, for example, methodologies described in Green et al., Molecular Cloning: A Laboratory Manual 4th. edition (2012) Cold Spring Harbor Laboratory Press, Cold Spring Harbor, N. Y.; and Ausubel, et al., Current Protocols in Molecular Biology, through Jul. 17, 2018, John Wiley & Sons, Inc. As appropriate, procedures involving the use of commercially available kits and reagents are generally carried out in accordance with manufacturer defined protocols and/or parameters unless otherwise noted. Before the present methods, expression systems, and uses therefore are described, it is to be understood that this invention is not limited to the particular methodology, protocols, cell lines, animal species or genera, constructs, and reagents described as such may, of course, vary. It is also to be understood that the terminology used herein is for the purpose of describing particular embodiments only, and is not intended to limit the scope of the present invention, which will be limited only by the appended claims.

### III. Cannabinoid Expression Systems

Cannabinoid compounds of interest and cannabinoid compound intermediates are produced using an expression system as described herein that employs a Type I or Type II PKS. Such compounds include, without limitation, CBG, CBDA, CBD, THC, Δ⁸-THC, THCA, Δ⁸-THCA, CBCA, CBA, CBN, CBDN, CBNA, CBV, CBVA, THCV, THCVA, Δ⁸-THCA, CBGV, CBGVA, CBCV, CBCVA, CBDV and CBDVA; as well as compounds including, but not limited to, the cannabichromanones, cannabicoumaronone, cannabicitran, 10-oxo-Δ^{6a(10a)}-tetrahydrohydrocannabinol (OTHC), cannabiglendol, and Δ⁷-isotetrahydrocannabinol, as well as analogs of such compounds, e.g., halogenated or deuterated compounds. In some embodiments, each step of a metabolic pathway that produces the cannabinoid compound of interests occurs in a modified recombinant cell described herein. In other embodiments, at least one step of the metabolic pathway occurs in a modified recombinant cell described herein, and at least one step of the metabolic pathway occurs extracellularly, e.g., in yeast media or within a co-cultured modified recombinant cell. The compounds produced at each step of the metabolic pathway may be referred to as “intermediates” or “intermediate compounds” or “compound intermediates”.

In one aspect, provided herein host cells for cannabinoid expression genetically modified to express an exogenous Type I or Type II PKS. In some embodiments, the host cells are additionally modified to express an exogenous polynucleotide that encodes an acyl-CoA synthetase that converts an aliphatic carboxylic acid to an acyl CoA thioester, e.g., a revS polypeptide, or alternatively, a CsAAE3, or CsAAE1 polypeptide, e.g., a transmembrane-domain-deleted CsAAE1 polypeptide; and in some embodiments, an exogenous polynucleotide that encodes a 2-alkyl-4,6-dihydroxybenzoic acid cyclase (e.g., olivetolic acid cyclase, including embodiments in which the olivetolic acid cyclase is truncated). In some embodiments, an acyl-CoA synthetase may comprise a deletion of a transmembrane domain.

In some embodiments, a genetically modified host cell expresses a Type I or Type II PKS that is modified to make cannabinoid precursors at high levels by substituting the native SAT and/or TE domains of PKSs that make short chain aromatic polyketides (such as 6-MSA or orsellinic acid) with SAT domains and/or TE domains from PKSs that naturally incorporate longer chain fatty acyl moieties such as PksA (see, e.g., Huitt-Roehl et al., ACS Chem Biol. 10:1443-1449, 2015) or the corresponding gene products of the micacocidin- or benastatin-producing gene clusters.

In further embodiments, additional constructs that encode cyclase enzymes are expressed in the same strains that express the PKSs. Such cyclase molecules may include, but are not restricted to, mutated C. sativa cyclase as described herein, AtHS1 and a BenH cyclase domain.

In some embodiments, the PKSs are modified orsellinic acid synthase (OSAS) enzymes, such as the orsA gene product of A. nidulans, or the OSAS of F. graminearum (PKS14). For example, in some embodiments, the SAT domain of the OrsA OSAS gene, or the SAT domain of the OSAS of F. graminearum, is replaced with the SAT domain of PksA (Huitt-Roehl et al., supra). In alternative embodiments, the SAT domain of OrsA OSAS or the SAT domain of the OSAS of F. graminearum, is replaced with BenQ. An illustrative OrsA OSAS amino acid sequence is provided in SEQ ID NO:20. The amino acid sequence of the illustrative SAT domain of OrsA is shown in SEQ ID NO:14. An illustrative F. graminearum OSAS sequence is provided in SEQ ID NO:15.

Additional embodiments include DNA constructs and their enzyme products derived from orsellinic acid, micacocidin- and benastatin-producing genes that are shuffled, in a directed manner, or through randomization of individual module genes from said gene clusters in order to biosynthesize, at high levels, cannabinoid and cannabinoid analog precursors.

### Cannibinoid Products

In some embodiments, a genetically modified host cell as described herein is used to produce a cannabinoid product, e.g., a halogenated or deuterated cannabinoid analog. For example, in some embodiments, starting material carboxylic acids such as 4-fluorobutanoic acid; 4,4,4-trifluorobutanoic acid; 2,2-difluorobutanoic acid; perfluorobutanoic acid; 5-fluoropentanoic acid; 2,2-difluoropentanoic acid; perfluoropentanoic acid; 6-fluorohexanoic acid; 2,2-difluorohexanoic acid; and perfluorohexanoic acid can be used in the preparation of cannabinoid analogs using a genetically modified host cell that expresses an exogenous Type I or Type II PKS as described herein.

In some embodiments, a carboxylic acid starting material according to Formula I is employed:

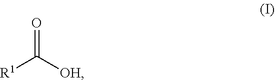

wherein R¹ is C₁-C₂₀ alkyl, C₁-C₂₀ haloalkyl, C₁-C₂₀ hydroxyalkyl, deuterated C₁-C₂₀ alkyl, tritiated C₁-C₂₀ alkyl, or C₂-C₂₀ alkenyl. In some embodiments, R¹ is selected from the group consisting of C₁-C₁₀ haloalkyl, C₁-C₁₀ hydroxyalkyl, deuterated C₁-C₁₀ alkyl, tritiated C₁-C₁₀ alkyl, or C₂-C₁₀ alkenyl. In some embodiments, the carboxylic acid is selected from the group consisting of 4-fluorobutanoic acid, 5-fluoropentanoic acid, and 6-fluorohexanoic acid.

In some embodiments, the methods include production of a 2-alkyl-4,6-dihydroxybenzoic acid 5- or alkylbenzene-1,3-diol according to Formula II:

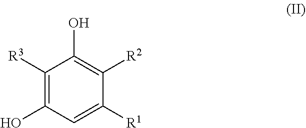

wherein:
R¹ is selected from the group consisting of C₁-C₂₀ alkyl, C₁-C₂₀ haloalkyl, C₁-C₂₀ hydroxyalkyl, deuterated C₁-C₂₀ alkyl, tritiated C₁-C₂₀ alkyl, and C₂-C₂₀ alkenyl,
R² is selected from the group consisting of COOR^{2a} and H,
R^{2a} is selected from the group consisting of H and C₁-C₆ alkyl, and
R³ is selected from the group consisting of a prenyl moiety and H.

In some embodiments, R¹ is selected from the group consisting of 4-chlorobutanoic acid, 4-bromobutanoic acid, 4-hydroxybutanoic acid, 5-chloropentanoic acid, 5-bromopentanoic acid, 5-hydroxypentanoic acid, 6-chlorohexanoic acid, 6-bromohexanoic acid, 6-hydroxyhexanoic acid, 7-chloroheptanoic acid, 7-bromoheptanoic acid, and 7-hydroxyheptanoic acid. In some embodiments, R¹ is perdeuterohexanoic acid (i.e., D₁₁C₅COOH).

In some embodiments, a genetically modified host cell expressing an exogenous Type I or Type II PKS can be employed for the production of a cannabinoid derivative compound. In some embodiments, the cannabinoid derivative is selected from a halogenated cannabidiolic acid, a halogenated cannabidiol, a halogenated Δ⁹-tetrahydrocannabinolic acid, a halogenated Δ⁸-tetrahydrocannabinolic acid, a halogenated cannabichromenic acid, a halogenated cannabichromene, a halogenated cannabinol, a halogenated cannabinodiol, a halogenated cannabinolic acid, a cannabivarin, a halogenated cannabivarinic acid, a halogenated Δ⁹-tetrahydrocannabivarin, a halogenated Δ⁸-tetrahydrocannabivarin, a halogenated Δ⁹-tetrahydrocannabivarinic acid, a halogenated Δ⁸-tetrahydrocannabivarinic acid, a halogenated cannabigerovarin, a halogenated cannabigerovarinic acid, a halogenated cannabichromevarin, a halogenated cannabichromevarinic acid, a halogenated cannabidivarin, a halogenated cannabidivarinic acid, a halogenated cannabitriol, and a halogenated cannabicyclol.

In some embodiments, the cannabinoid derivative is selected from a deuterated cannabidiolic acid, a deuterated cannabidiol, a deuterated Δ⁹-tetrahydrocannabinolic acid, a deuterated Δ⁸-tetrahydrocannabinolic acid, a deuterated cannabichromenic acid, a deuterated cannabichromene, a deuterated cannabinol, a deuterated cannabinodiol, a deuterated cannabinolic acid, a cannabivarin, a deuterated cannabivarinic acid, a deuterated Δ⁹-tetrahydrocannabivarin, a deuterated Δ⁸-tetrahydrocannabivarin, a deuterated Δ⁹-tetrahydrocannabivarinic acid, a deuterated Δ⁸-tetrahydrocannabivarinic acid, a deuterated cannabigerovarin, a deuterated cannabigerovarinic acid, a deuterated cannabichromevarin, a deuterated cannabichromevarinic acid, a deuterated cannabidivarin, a deuterated cannabidivarinic acid, a deuterated cannabitriol, and a deuterated cannabicyclol.

In some embodiments, the cannabinoid derivative is selected from a tritiated cannabidiolic acid, a tritiated cannabidiol, a tritiated Δ⁹-tetrahydrocannabinolic acid, a tritiated Δ⁸-tetrahydrocannabinolic acid, a tritiated cannabichromenic acid, a tritiated cannabichromene, a tritiated cannabinol, a tritiated cannabinodiol, a tritiated cannabinolic acid, a cannabivarin, a tritiated cannabivarinic acid, a tritiated Δ⁹-tetrahydrocannabivarin, a tritiated Δ⁸-tetrahydrocannabivarin, a tritiated Δ⁹-tetrahydrocannabivarinic acid, a tritiated Δ⁸-tetrahydrocannabivarinic acid, a tritiated cannabigerovarin, a tritiated cannabigerovarinic acid, a tritiated cannabichromevarin, a tritiated cannabichromevarinic acid, a tritiated cannabidivarin, a tritiated cannabidivarinic acid, a tritiated cannabitriol, and a tritiated cannabicyclol.

In some embodiments, the cannabinoid derivative is selected from a hydroxy-cannabidiolic acid, a hydroxy-cannabidiol, a hydroxy-Δ⁹-tetrahydrocannabinolic acid, a hydroxy-Δ⁸-tetrahydrocannabinolic acid, a hydroxy-cannabichromenic acid, a hydroxy-cannabichromene, a hydroxy-cannabinol, a hydroxy-cannabinodiol, a hydroxy-cannabinolic acid, a cannabivarin, a hydroxy-cannabivarinic acid, a hydroxy-Δ⁹-tetrahydrocannabivarin, a hydroxy-Δ⁸-tetrahydrocannabivarin, a hydroxy-Δ⁹-tetrahydrocannabivarinic acid, a hydroxy-Δ⁸-tetrahydrocannabivarinic acid, a hydroxy-cannabigerovarin, a hydroxy-cannabigerovarinic acid, a hydroxy-cannabichromevarin, a hydroxy-cannabichromevarinic acid, a hydroxy-cannabidivarin, a hydroxy-cannabidivarinic acid, a hydroxy-cannabitriol, and a hydroxy-cannabicyclol.

In some embodiments, cannabinoid products set forth in Table 1 can be prepared using chemical steps and/or cannabinoid synthase-catalyzed steps, as described below.

TABLE 1
Cannabinoid Products

| Cannabinoid derivative structure | Derivative name |
|---|---|
| 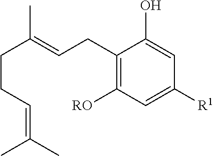 | cannabigerol [CBG] analog (R = H) cannabigerol monomethyl ether [CBGM] analog (R = CH₃) cannabigerovarin [CBGV] analog |
| 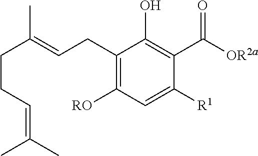 | cannabigerolic acid A [CBGA] analog (R = H) cannabigerolic acid A monomethyl ether [CBGAM] analog (R = CH₃) cannabigerovarinic acid [CBGVA] analog |
| 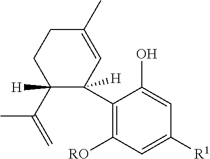 | (−)-cannabidiol [CBD] analog (R = H) cannabidiol monomethyl ether [CBDM] analog (R = CH₃) cannabidivarin [CBDV] analog cannabidiorcol [CBD-C1] analog |
| 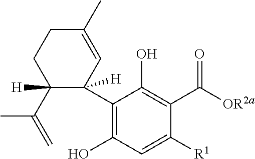 | cannabidiolic acid [CBDA] analog cannabidivarinic acid [CBDVA] analog |
| 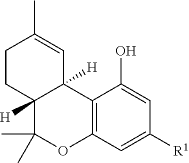 | Δ⁹-tetrahydrocannabinol [THC] analog Δ⁹-tetrahydrocannabivarin [THCV] analog Δ⁹-tetrahydrocannabiorcol [THC-C₁] analog |
| 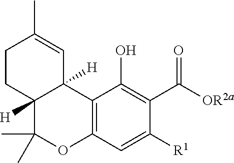 | Δ⁹-tetrahydrocannabinolic acid [Δ⁹-THCA] analog Δ⁹-tetrahydrocannabivarinic acid [Δ⁹-THCVA] analog Δ⁹-tetrahydrocannabiorcolic acid [THCOA] analog |
| 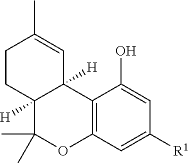 | (−)-(6aS,10aR)-Δ⁹-tetrahydrocannabinol [cis-Δ⁹-THC] analog |
| 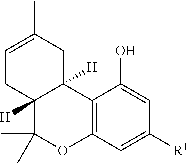 | (−)-Δ⁸-trans-(6aR,10aR)-Δ⁸-Δ⁸-tetrahydrocannabinol [Δ⁸-THC] analog (−)-Δ⁸-trans-(6aR,10aR)-Δ⁸-Δ⁸-tetrahydrocannabivarin [Δ⁸-THCV] analog |
| 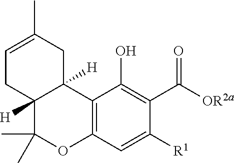 | (−)-Δ⁸-trans-(6aR,10aR)-Δ⁸-tetrahydrocannabinolic acid [Δ⁸-THCA] analog Δ⁸-tetrahydrocannabivarinic acid [Δ⁸-THCVA] analog |
| 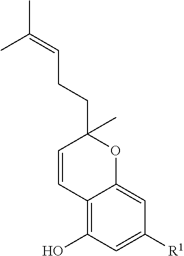 | cannabichromene [CBC] analog cannabichromevarin [CBCV] analog |
| 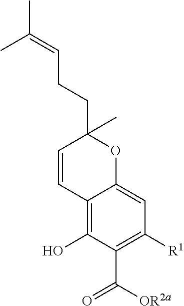 | cannabichromenic acid [CBCA] analog cannabichromevarinic acid [CBCVA] analog |
| 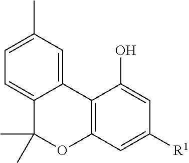 | cannabinol [CBN] analog cannabinol methyl ether [CBNM] analog cannabivarin [CBV] analog cannabiorcol [CBN-C₁] analog |
| 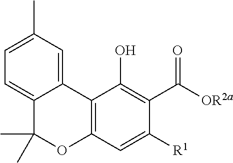 | cannabinolic acid [CBNA] analog cannabivarinic acid [CBVA] analog |
| 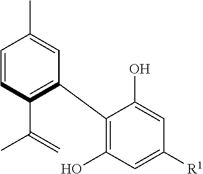 | cannabinodiol [CBND] analog cannabinodivarin [CBND-C3] analog |
| 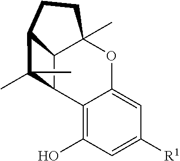 | (±)-(1aS,3aR,8bR,8cR)-cannabicyclol [CBL] analog (±)-(1aS,3aR,8bR,8cR)-cannabicyclovarin [CBLV] analog |
| 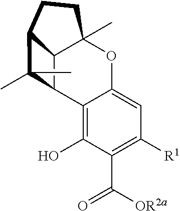 | (±)-(1aS,3aR,8bR,8cR)-cannabicyclolic acid [CBLA] analog |
| 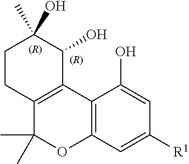 | (−)-(9R,10R)-trans-cannabitriol [(−)-trans-CBT] analog |
| 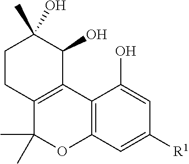 | (+)-(9S,10S)-trans-cannabitriol [(+)-trans-CBT] analog |
| 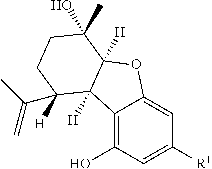 | (5aS,6S,9R,9aR)-cannabielsoin [CBE] analog |
| 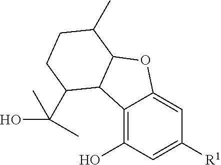 | cannabiglendol-C₃ [OH-iso-HHCV-C₃] analog |
| 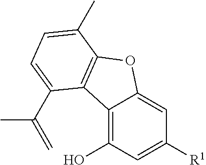 | dehydrocannabifuran [DCBF] analog |
| 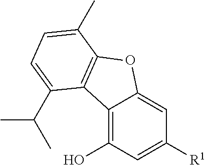 | cannabifuran [CBF] analog |
| 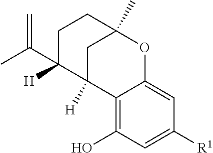 | (−)-Δ⁷-trans-(1R,3R,6R)-isotetrahydrocannabinol analog (−)-Δ⁷-trans-(1R,3R,6R)-isotetrahydrocannabivarin |
| 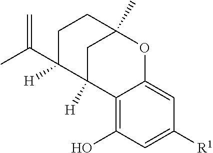 | (±)-Δ⁷-1,2-cis-(1R,3R,6S)-isotetrahydrocannabivarin analog |
| 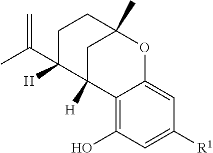 | (±)-Δ⁷-1,2-cis-(1S,3S,6R)-isotetrahydrocannabivarin analog |
| 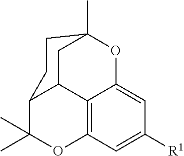 | cannabicitran [CBT] analog |
| 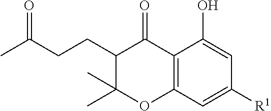 | cannabichromanone [CBCN] analog |
| 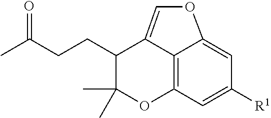 | cannabicoumaronone [CBCON] analog |

Cannabinoid products include, without limitation, CBG, CBDA, CBD, THC, Δ⁸-THC, THCA, Δ⁸-THCA, CBCA, CBC, CBN, CBND, CBNA, CBV, CBVA, THCV, THCVA, Δ⁸-THCA, CBGV, CBGVA, CBCV, CBCVA, CBDV and CBDVA, as well as analogs thereof. Further examples include, but are not limited to, the cannabichromanones, cannabicoumaronone, cannabicitran, 10-oxo-Δ^{6a(10a)}-tetrahydrohydrocannabinol (OTHC), cannabiglendol, and Δ⁷-isotetrahydrocannabinol.

In some embodiments, cannabinoid products as set forth in Table 1 are provided, wherein R¹ is selected from the group consisting of C₁-C₁₀ alkyl, C₁-C₁₀ haloalkyl, C₁-C₁₀ hydroxyalkyl, deuterated C₁-C₁₀ alkyl, tritiated C₁-C₁₀ alkyl, and C₂-C₁₀ alkenyl.

### Type I PKS

In some embodiments, a host cell is genetically modified to express an exogenous polynucleotide that encodes a Type I PKS or a non-naturally occurring variant of a Type I PKS that has polyketide synthase activity. In some embodiments, the Type I PKS is an iterative partially reducing PKS. Partially reducing PKSs share a highly conserved domain architecture that distinguishes them from non-reducing and highly reducing PKSs in that although they may have a ketoreductase (KR) domain, they lack dehydratase or enoylreductase domains for further reductive processing. In some embodiments, Type I PKS polypeptides are selected to employ hexanoyl-CoA as a starter unit.

Type I PKSs that can be preferentially utilized include PKSs that are naturally initiated by a starter unit hexanoyl-CoA such as the PKS encoding the micacocidin biosynthetic pathway or, alternatively, iterative Type I PKSs such as orsellinic acid synthase (OSAS), or 6-methylsalicylic acid synthase (6-MSAS) that have been mutated to accept longer chain fatty acid starter units to produce olivetolic and divarinic acids and their analogs.

In exemplary embodiments, the exogenous Type I PKS is an iterative partially reducing PKS that produces the antibiotic micacocidin and is derived from the bacterium Ralstonia solanacearum (Kage et al., Chemistry and Biology 20:764-771, 2013; Kage et al., Org. Biomol. Chem. 13:11414-11417, 2015).

The MicC PKS of Ralstonia solanacearum comprises a loading module followed by three extender modules. In some embodiments of a genetically modified host cell as described herein, the Type I PKS encoded by an exogenous polynucleotide comprises the loading module and extender module 1 of MicC, which comprises the following domains: an adenylation (A₁) domain, an acyl carrier protein (ACP) domain, a ketosynthase (KS) domain, an acyl transferase (AT) domain, a KR domain, and an ACP domain at the C-terminal end of the module. In some embodiments, the PKS comprises a MicC polypeptide sequence, e.g., as set forth in SEQ ID NO:2. In some embodiments, the KR domain is inactivated by mutation at the active site of the KR domain, e.g., by mutation of the Tyr at position 1991, which is part of a catalytic triad together with Lys and Ser residues (see, e.g., Caffrey, Chem Bio Chem 4:654-657, 2003). In some embodiments, a phenylalanine is introduced to substitute for the Tyr at position 1991. In other embodiments, an aliphatic amino acid residues, e.g., alanine, is substituted for Tyr at position 1991.

In some embodiment the exogenous polynucleotide encodes a Type I PKS that comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:1. In some embodiments, the polynucleotide encodes a Type I PKS polypeptide that has at least 70%, 75%, 80%, 85%, 90%, 95%, or greater, identity to the sequence set forth in SEQ ID NO:1. In some embodiments, the Type I PKS comprises a polypeptide sequence that is a non-naturally occurring variant of SEQ ID NO:1. In some embodiments, the variant comprises a mutation in the KR domain that inactivates the KR domain. In some embodiments, the PKS comprises a polypeptide sequence as set forth in SEQ ID NO:1 in which the Tyrosine at positions 1991, as determined with reference to SEQ ID NO:1, comprises a substitution, e.g., an alanine substitutions that inactivates the KR domain.

In some embodiments, the genetically modified host cell is further engineered to express a phosphopantetheinyl transferase (PPTase). In particular embodiments, the PPTase gene is MicA from Ralstonia solanacearum, or an ortholog thereof, e.g., from another Ralstonia species. In some embodiments, the PPTase comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:2. In some embodiments, the polynucleotide encodes a PPTase that has at least 70%, 75%, 80%, 85%, 90%, 95%, or greater, identity to the sequence set forth in SEQ ID NO:2. In some embodiments, the PPTase comprises the amino acid sequence of SEQ ID NO:2. In alternative embodiments, the PPTase is a fungal or bacterial PPTase, e.g., NpgA or sfp.

In some embodiments the Type I PKS is a mutant orsellinic acid synthase derived from Aspergillus nidulans (orsA) or from Fusarium graminearum (PKS14). For example, the SAT domain of the OSAS Orsa or of PKS14 can be replaced with the SAT domain of PksA or BenQ.

### Type II PKS

In some embodiments, a host cell is genetically modified to express an exogenous polynucleotide that encodes a Type II PKS or a non-naturally occurring variant of a Type II PKS that has polyketide synthase activity. In some embodiments, the Type II PKS encodes a PKS that can use hexnoyl coA as a starter unit. In some embodiments, the Type II PKS comprises a BenA polypeptide or a multimeric BenA-BenB-BenC PKS enzyme from a Streptomyces sp., or an ortholog thereof, that naturally produces benastatin. As used herein, a “BenA PKS” refers to a PKS comprising BenA encoded by the BenA gene of the benastatin gene cluster. In some embodiments, a “BenA PKS” additionally contains BenB and BenC.

In some embodiment the exogenous polynucleotide encodes a Type II PKS that comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:3. In some embodiments, the polynucleotide encodes a Type II PKS polypeptide that has at least 70%, 75%, 80%, 85%, 90%, 95%, or greater, identity to the sequence set forth in SEQ ID NO:3. In some embodiments, the Type II PKS comprises a polypeptide sequence that is a non-naturally occurring variant of SEQ ID NO:3.

In some embodiments, the genetically modified host cell is further engineered to express BenQ, a FabH-like ketoacyl-synthase (KASIII), which plays a role in providing and selecting hexanoate as the PKS starter unit. In particular embodiments, the polynucleotide introduced in the genetically modified host cell comprises a nucleic acid sequence that encodes BenQ from a Streptomyces sp, or an ortholog thereof. In some embodiments, the BenQ polypeptide comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:4. In some embodiments, the polynucleotide encodes a BenQ polypeptide that has at least 70%, 75%, 80%, 85%, 90%, 95%, or greater, identity to the sequence set forth in SEQ ID NO:4. In some embodiments, the BenQ polypeptide comprises the amino acid sequence of SEQ ID NO:4.

In some embodiments, the host cell is genetically modified to express a multimeric BenA-BenB-BenC PKS enzyme. In some embodiments, the polynucleotide introduced in the genetically modified host cell comprises a nucleic acid sequence that encodes BenB from a Streptomyces sp, or an ortholog thereof. In some embodiments, the BenB polypeptide comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:17. In some embodiments, the polynucleotide encodes a BenB polypeptide that has at least 70%, 75%, 80%, 85%, 90%, 95%, or greater, identity to the sequence set forth in SEQ ID NO:17. In some embodiments, the BenB polypeptide comprises the amino acid sequence of SEQ ID NO:4. In further embodiments, the polynucleotide introduced in the genetically modified host cell comprises a nucleic acid sequence that encodes BenC from a Streptomyces sp, or an ortholog thereof. In some embodiments, the BenC polypeptide comprises an amino acid sequence that has at least 60% or greater, identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity) to the sequence set forth in SEQ ID NO:18. In some embodiments, the polynucleotide encodes a BenC polypeptide that has at least 70%, 75%, 80%, 85%, 90%, 95%, or greater, identity to the sequence set forth in SEQ ID NO:18. In some embodiments, the BenC polypeptide comprises the amino acid sequence of SEQ ID NO:18.

### 2-Alkyl-4,6-dihydroxybenzoic Acid Cyclase

A host cell in accordance with the invention may be further modified to express an exogenous polynucleotide that encodes a 2-alkyl-4,6-dihydroxybenzoic acid cyclase (e.g., olivetolic acid cyclase). In some embodiments, the 2-alkyl-4,6-dihydroxybenzoic acid cyclase is a dimeric α+β barrel (DABB) protein domain that resembles DABB-type polyketide cyclases from Streptomyces. Olivetolic acid cyclase is described, for example, by Gagne et al. (Proc. Nat. Acad. Sci. USA 109 (31): 12811-12816; 2012). The term “2-alkyl-4,6-dihydroxybenzoic acid cyclase” includes variants, e.g., a truncated or modified polypeptide, that have cyclase activity; and naturally occurring homologs or orthologs. In some embodiments, the 2-alkyl-4,6-dihydroxybenzoic acid cyclase is olivetolic acid cyclase from C. sativa (EC number 4.4.1.26). In some embodiments, the 2-alkyl-4,6-dihydroxybenzoic acid cyclase produces divarinic acid (see, e.g., Yang et al., FEBS J. 283:1088-1106, 2016). In some embodiments, the 2-alkyl-4,6-dihydroxybenzoic acid cyclase is an olivetolic acid cyclase homolog from Arabidopsis thaliana AtHS1 (Uniprot Q9LUV2, see also Yang et al., supra), Populus tremula SP (P0A881), A. thaliana At5g22580 (Q9FK81), S. glaucescens TcmI cyclase (P39890), S. coelicolor ActVA-Orf6 (Q53908), P. reinekei MLMI (C5MR76), S. nogalater SnoaB (O54259), M. tuberculosis Rv0793 (O86332), or P. aeruginosa PA3566 (Q9HY51). In some embodiments, the cyclase is the N-terminal domain of a BenH protein from a benastatin gene cluster, e.g., from Streptomyces sp. A2991200. In some embodiments, the 2-alkyl group of the 2-alkyl-4,6-dihydroxybenzoic acid contains 1-18 carbon atoms. In some embodiments, the 2-alkyl group of the 2-alkyl-4,6-dihydroxybenzoic acid contains 1-12 carbon atoms. In some embodiments, the 2-alkyl group of the 2-alkyl-4,6-dihydroxybenzoic acid contains 1-9 carbon atoms.

In some embodiments, the polynucleotide encoding the 2-alkyl-4,6-dihydroxybenzoic acid cyclase encodes a polypeptide that has 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:8, 9, or 10. In some embodiments, the polypeptide has at least 70%, 75%, 80%, 85%, 90%, 95%, or greater identity to the sequence set forth in SEQ ID NO:8, 9, or 10.

In some embodiments, the polynucleotide encoding the 2-alkyl-4,6-dihydroxybenzoic acid cyclase encodes an a polypeptide has 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:12. In some embodiments, the polypeptide has at least 70%, 75%, 80%, 85%, 90%, 95%, or greater identity to the sequence set forth in SEQ ID NO:12.

In some embodiments, the polynucleotide encoding the 2-alkyl-4,6-dihydroxybenzoic acid cyclase encodes an a polypeptide has 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:13. In some embodiments, the polypeptide has at least 70%, 75%, 80%, 85%, 90%, 95%, or greater identity to the sequence set forth in SEQ ID NO:13.

### Acyl-CoA Synthetase

In some embodiments, the host cell is genetically modified to express an acyl-CoA synthetase, which may also be referred to herein as an “acyl-CoA synthase”, an “acyl activating enzyme”, or an “acyl-CoA ligase”, is an enzyme that in the present invention converts an aliphatic carboxylic acid to an acyl-CoA thioester through a two-step process in which a carboxylate and ATP are converted to an enzyme-bound carboxyl-AMP intermediate (called an adenylate) with the release of pyrophosphate (PPi). The activated carbonyl carbon of the adenylate is coupled to the thiol of CoA, followed by enzyme release of the thioester and AMP. Any number of acyl-CoA synthetases can be employed in the present invention. Acyl-CoA synthetases include, but are not limited to, short-chain acyl-CoA synthetases (EC 6.2.1.1), medium chain acyl-CoA synthetases (EC 6.2.1.2), long-chain acyl-CoA synthetases (EC 6.2.1.3), and coumarate-CoA ligases (EC 6.2.1.12). Acyl-CoA synthetases typically include a 12-amino acid residue domain called the AMP-binding motif (PROSITE PS00455): [LIVMFY]-{E}-{VES}-[STG]-[STAG]-G-[ST]-[STEI]-[SG]-x-[PASLIVM]-[KR]. In the PROSITE sequence, each position in the sequence is separated by “-” and the symbol “x” means that any residue is accepted at the given location in the sequence. Acceptable amino acids for a given position are placed between square parentheses (e.g., [ST] indicates that serine or threonine are acceptable at the given location in the sequence), while amino acids which are not accepted at a given location are placed between curly brackets (e.g., {VES} indicates that any residue except valine, glutamic acid, and serine are acceptable at the given location in the sequence). The AMP binding motif has been used to classify polypeptides as acyl activating enzymes (AAEs) and contributed to the identification of the large AAE gene superfamily present in Arabidopsis (Shockey et al., Plant Physiology 132:1065-1076, 2003), Chlamydomonas reinhardtii, Populus trichocharpa, and Physcomitrella patens (Shockey and Browse, The Plant Journal (2011) 66:143-160, 2011). Acyl-CoA synthetases are also described, for example, by Black et al. (Biochim Biophys Acta. 1771(3):286-98, 2007); Miyazawa et al. (J. Biol. Chem 290 (45): 26994-27011, 2015); and Stout et al. (Plant J. 71(3):353-365, 2012). In some embodiments, the acyl-CoA synthetase is from an organism that biosynthesizes resveratrol. In some embodiments, the acyl-CoA synthetase is a coumarate-CoA ligase from the genus Morus or the genus Vitis. In some embodiments, the acyl-CoA synthetase is from Ralstonia solanacearum. In some embodiments, the acyl-CoA synthetase from Ralstonia solanacearum is deleted at the N-terminus, see, e.g., SEQ ID NO:11.

In some embodiments, a host cell is genetically modified to express an exogenous polynucleotide that encodes a revS polypeptide from a Streptomyces sp. (see, e.g., Miyazawa et al., J. Biol. Chem. 290:26994-27001, 2015), or variant thereof, e.g., a native homolog, ortholog or non-naturally occurring variant that has acyl-CoA synthetase activity. In some embodiments, the polynucleotide encodes a polypeptide that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:. In some embodiments, the polynucleotide encodes a RevS polypeptide that has about 70%, 75%, 80%, 85%, 90%, 95%, or greater identity to the sequence set forth in SEQ ID NO:5. In some embodiments, a non-naturally occurring variant comprises one or more modifications, e.g., substitutions such as conservative substitutions, in comparison to SEQ ID NO:5, e.g., in regions outside the AMP binding motif or catalytic site.

In some embodiments, a host cell is genetically modified to express an exogenous polynucleotide that encodes an acyl activating enzyme from Cannabis sativa (CsAAE3) or variant thereof, e.g., a native homolog, ortholog or non-naturally occurring variant that has acyl-CoA synthetase activity. In some embodiments, the CsAAE3 polypeptide encoded by the polynucleotide comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:6. In some embodiments, the acyl-CoA synthetase polynucleotide encodes a CsAAE3, or a homolog or non-naturally occurring thereof, comprising an amino acid sequence that has at least 70%, 75%, 80%, 85%, 90%, 95%, or greater identity to the sequence set forth in SEQ ID NO:6. In some embodiments, a non-naturally occurring variant comprises one or more modifications, e.g., substitutions such as conservative substitutions, in comparison to SEQ ID NO:6, e.g., in regions outside the AMP binding motif or catalytic site.

In some embodiments, a host cell is genetically modified to express an exogenous polynucleotide that encodes an acyl activating enzyme from Cannabis sativa (CsAAE1) or variant thereof, e.g., a native homolog, ortholog or non-naturally occurring variant that has acyl-CoA synthetase activity. In some embodiments, the CsAAE1 polypeptide encoded by the polynucleotide comprises an amino acid sequence that has at least 60% or greater identity (e.g., at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity) to the sequence set forth in SEQ ID NO:7. In some embodiments, the acyl-CoA synthetase polynucleotide encodes a CsAAE1, or a homolog thereof, comprising an amino acid sequence that has at least 70%, 75%, 80%, 85%, 90%, 95%, or greater identity to the sequence set forth in SEQ ID NO:7. In some embodiments, the CsAAE1 polynucleotide encodes a polypeptide from which the transmembrane domain is deleted. In some embodiments, a non-naturally occurring variant comprises one or more modifications, e.g., substitutions such as conservative substitutions, in comparison to SEQ ID NO:7, e.g., in regions outside the AMP binding motif or catalytic site.

The acyl-CoA synthetase can be used in conjunction with a number of aliphatic carboxylic acid starting materials including, but not limited to, butanoic acid (butyric acid), pentanoic acid (valeric acid), hexanoic acid (caproic acid), heptanoic acid (enanthic acid), and octanoic acid (caprylic acid). In some embodiments, hexanoic acid is used for formation of hexanoyl-CoA by the acyl-CoA synthetase.

### Chemical Thioester Synthesis

In some embodiments, a chemically-synthesized thioester is used as a starting material instead of employing an acyl-CoA synthetase to enzymatically produce the thioester from a carboxylic acid.

For example, a thioester according to Formula II

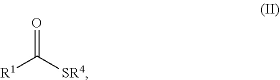

may contain a CoA R⁴ moiety, a pantetheine R⁴ moiety, or a cysteamine R⁴ moiety. A thioester according to Formula II can be prepared enzymatically using an acyl-CoA synthetase expressed by the host cell as described above, or the thioester can be synthesized by chemically acylating CoA, pantetheine (i.e., 2,4-dihydroxy-3,3-dimethyl-N-[2-(2-sulfanylethylcarbamoyl)ethyl]butanamide), or cysteamine (i.e., 2-aminoethanethiol) with a carboxylic acid according to Formula I or an activated derivative thereof. In some embodiments, R¹ may be an unsubstituted alkyl group. In some embodiments, R¹ may be a C₁-C₁₀ haloalkyl group, a C₁-C₁₀ hydroxyalkyl group, a deuterated C₁-C₁₀ alkyl group, a tritiated C₁-C₁₀ alkyl group, or a C₂-C₁₀ alkenyl group.

A carboxylic acid according to Formula I can be used in conjunction with a coupling agent for acylation of the thiol to be acylated (e.g., CoA, pantetheine, or cysteamine). Coupling agents include for example, carbodiimides (e.g., N,N′-dicyclohexylcarbodiimide (DCC), N,N′-dicyclopentylcarbodiimide, N,N′-diisopropylcarbodiimide (DIC), 1-ethyl-3-(3-dimethylaminopropyl)carbodiimide (EDC), etc.), phosphonium salts (HOBt, PyBOP, HOAt, etc.), aminium/uronium salts (e.g., pyrimidinium uronium salts such HATU, tetramethyl aminium salts, bispyrrolidino aminium salts, bispiperidino aminium salts, imidazolium uronium salts, uronium salts derived from N,N,N′-trimethyl-N′-phenylurea, morpholino-based aminium/uronium coupling reagents, antimoniate uronium salts, etc.), organophosphorus reagents (e.g., phosphinic and phosphoric acid derivatives), organosulfur reagents (e.g., sulfonic acid derivatives), triazine coupling reagents (e.g., 2-chloro-4,6-dimethoxy-1,3,5-triazine, 4-(4,6-dimethoxy-1,3,5-triazin-2-yl)-4 methylmorpholinium chloride, 4-(4,6-dimethoxy-1,3,5-triazin-2-yl)-4 methylmorpholinium tetrafluoroborate, etc.), pyridinium coupling reagents (e.g., Mukaiyama's reagent, pyridinium tetrafluoroborate coupling reagents, etc.), polymer-supported reagents (e.g., polymer-bound carbodiimide, polymer-bound TBTU, polymer-bound 2,4,6-trichloro-1,3,5-triazine, polymer-bound HOBt, polymer-bound HOSu, polymer-bound IIDQ, polymer-bound EEDQ, etc.), and the like.

Alternatively, acylation can be conducted using an activated carboxylic acid derivative such as an acid anhydride, a mixed anhydride an acid chloride, or an activated ester (e.g., a pentafluorophenyl ester or an N-hydroxysuccinimidyl ester). Typically, 1-10 molar equivalents of the carboxylic acid or activated derivative with respect to the thiol will be used. For example, 1-5 molar equivalents of the acid/acid derivative or 1-2 molar equivalents of the acid/acid derivative can be used. In some embodiments, around 1.0, 1.1, 1.2, 1.3, 1.4, or 1.5 molar equivalents of the acid/acid derivative with respect to the thiol is used to form the thioester according to Formula II.

A base can be used to promote acylation of the thiol by the carboxylic acid or the activated carboxylic acid derivative. Examples of suitable bases include potassium carbonate, sodium carbonate, sodium acetate, Huenig's base (i.e., N,N-diisopropylethylamine), lutidines including 2,6-lutidine (i.e., 2,6-dimethylpyridine), triethylamine, tributylamine, pyridine, 2,6-di-tert-butylpyridine, 1,8-diazabicycloundec-7-ene (DBU), quinuclidine, and the collidines. Combinations of two or more bases can be used. Typically, less than one molar equivalent of base with respect to the thiol will be employed in the formation of the thioester. For example, 0.05-0.9 molar equivalents or 0.1-0.5 molar equivalents of the base can be used. In some embodiments, around 0.05, 0.1, 0.15, or 0.2 molar equivalents of the base with respect to the thiol is used in conjunction with the acid/acid derivative to form the thioester according to Formula II.

Any suitable solvent can be used for forming the thioester. Suitable solvents include, but are not limited to, toluene, methylene chloride, ethyl acetate, acetonitrile, tetrahydrofuran, benzene, chloroform, diethyl ether, dimethyl formamide, dimethyl sulfoxide, petroleum ether, and mixtures thereof. The acylation reaction is typically conducted at temperatures ranging from around 25° C. to about 100° C. for a period of time sufficient to form the thioester according to Formula II. The reaction can be conducted for a period of time ranging from a few minutes to several hours or longer, depending on the particular thiol and acid/acid derivative used in the reaction. For example, the reaction can be conducted for around 10 minutes, or around 30 minutes, or around 1 hour, or around 2 hours, or around 4 hours, or around 8 hours, or around 12 hours at around 40° C., or around 50° C., or around 60° C., or around 70° C., or around 80° C.

Functional groups such as the primary amine of cysteamine or the hydroxyl groups of pantetheine and CoA can be protected to prevent unwanted side reactions during the acylation step. Examples of amine protecting groups include, but are not limited to, benzyloxycarbonyl; 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; and the like. Examples of hydroxyl protecting groups include, but are not limited to, benzyl; tert-butyl; trityl; tert-butyldimethylsilyl (TBDMS; TBS); 4,5-dimethoxy-2-nitrobenzyloxycarbonyl (Dmnb); propargyloxycarbonyl (Poc); and the like. Other alcohol protecting groups and amine protecting groups are known to those of skill in the art including, for example, those described by Green and Wuts (Protective Groups in Organic Synthesis, 4^{th} Ed. 2007, Wiley-Interscience, New York). The protecting groups can be removed using standard conditions so as to restore the original functional groups following the acylation step.

### Additional Modifications

In some embodiments, a recombinant host cell engineered to express an acyl-CoA synthetase; a Type I or Type II PKS synthase, e.g., a MicC or BenA polypeptide; and a 2-alkyl-4,6-dihydroxybenzoic acid cyclase, may be further modified to express an exogenous polynucleotide that encodes a prenyltransferase that catalyzes coupling of geranyl-pyrophosphate to a 2-alkyl-4,6-dihydroxybenzoic acid (e.g., olivetolic acid) to produce acidic cannabinoids such as cannabigerolic acid (CBGA). Examples of prenyltransferases include geranylpyrophosphate:olivetolate geranyltransferase (GOT; EC 2.5.1.102) as described by Fellermeier & Zenk (FEBS Letters 427:283-285; 1998). Streptomyces prenyltransferases including NphB, as described by Kumano et al. (Bioorg Med Chem. 16(17): 8117-8126; 2008), can also be used in accordance with the invention. In some embodiments, the prenyltransferase is fnq26, i.e., flaviolin linalyltransferase from Streptomyces cinnamonensis. In some embodiments, a host cell genetically modified to express the prenyltransferase may be a modified host cell as described in the following below.

Exogenous prenyl species, such as geraniol, can be supplied to the host cells during culture and production of the prenylated compounds. Alternatively, the host cells can be cultured in media containing high levels of prenyl precursors, e.g., prenol, isoprenol, geraniol, and the like. In procedures including multiple precursor feeding (MPF), 5-carbon prenol and isoprenol can be enzymatically converted to the monophosphate level (i.e., to dimethylallyl monophosphate and isopentenyl monophosphate) and then to the diphosphate level (i.e., to dimethylallyl pyrophosphate and isopentenyl pyrophosphate) prior to coupling to form the 10-carbon geranyl pyrophosphate.

Thus, as detailed herein, in some embodiments relating to the biosynthesis of an initiating aromatic polyketide precursor, enzymes that form simple starting units are expressed and used to generate, from exogenously supplied aliphatic carboxylic acids, acylthioesters, typically acetyl-, propionyl-, butanoyl-, hexanoyl-, malonyl- or methylmalonyl-coenzyme-A (CoA) thioesters. These are then condensed repeatedly with malonyl-CoA to form the aromatic polyketide building blocks for the next step in cannabinoid biosynthesis, namely prenylation.

In some embodiments, the starting carboxylic acids is hexanoic acid or butanoic acid, giving rise to precursors for the eventual production of cannabigerolic or cannabinogerovarinic acid-type molecules, and their decarboxylated, and otherwise chemically transformed, derivatives.

In some embodiments, modified recombinant host cells are also provided, which host cells comprise an exogenous polynucleotide that encodes prenol and isoprenol kinase; an exogenous polynucleotide that encodes kinase activity to produce dimethylallyl pyrophosphate and isopentenyl pyrophosphate when grown in the presence of exogenous prenol and isoprenol; an exogenous polynucleotide that encodes a geranyl-pyrophosphate synthase; and and/or an exogenous polynucleotide that encodes a prenyltransferase that catalyzes coupling of geranyl-pyrophosphate to olivetolic acid or an olivetolic acid analog (e.g., a 2-alkyl-4,6-dihydroxybenzoic acid) to form a cannabinoid compound. In some embodiments, the 2-alkyl group of the 2-alkyl-4,6-dihydroxybenzoic acid contains 1-18 carbon atoms. In some embodiments, the 2-alkyl group of the 2-alkyl-4,6-dihydroxybenzoic acid contains 1-12 carbon atoms. In some embodiments, the 2-alkyl group of the 2-alkyl-4,6-dihydroxybenzoic acid contains 1-9 carbon atoms.

Five-carbon prenols (prenol and isoprenol) may be converted by several enzymes to the monophosphate level and then to the diphosphate level by additional expressed enzymes, prior to their coupling to give the 10-carbon geranyl-diphosphate by the enzyme GPP-synthase. In some embodiments, the initial kinase event is performed by the enzyme hydroxyethylthiazole kinase. This enzyme has been described in several organisms from where the encoding genes are derived, including E. coli, Bacillus subtilis, Rhizobium leguminosarum, Pyrococcus horikoshii, S. cerevisiae and maize species.

Further phosphorylation to the diphosphate level is achieved by using the enzyme isoprenyl diphosphate synthase or isopentenylphosphate kinase, see U.S. Pat. No. 6,235,514. In some embodiments, chemically synthesized genes encoding this enzyme or more active mutants are derived by using the Thermoplasma acidophilum, Methanothermobacter thermautotrophicus, Methano-caldococcus jannaschii, Mentha x pperita or Mangifera indica amino acid sequences, or other homologous sequences with kinase activity.

The 10-carbon geranyl-diphosphate may also be generated by a kinase that phosphorylates geraniol to the monophosphate level, followed by a second kinase that gives rise to geranyl-diphosphate. In some embodiments, the first kinase event is performed by the enzyme farnesol kinase (FOLK) (Fitzpatrick, Bhandari and Crowell, 2011; Plant J. 2011 June; 66(6):1078-88). This kinase enzyme is derived from the known amino acid sequences or mutants from the organisms that phosphorylate the 5-carbon prenols, including plants (Arabidopsis thaliana, Camelina sativa, Capsella rubella, Noccaea caerulescens etc.) and fungi (Candida albicans, Talaromyces atroroseus, etc.).

Further phosphorylation of geranyl-phosphate to the geranyl-diphosphate level is achieved by using a mutated enzyme isopentenyl monophosphate kinase (IPK) Mutations in IPK (Val73, Val130, Ile140) have been reported to give rise to enhanced geranyl-phosphate kinase activity (Mabanglo et al., 2012). This kinase enzyme is derived from the known amino acid sequences or mutants from bacteria or archaeal species, including but not limited to Methanocaldococcus jannaschii, and Thermoplasma acidophilum.

In some embodiments, the DNA construct for the prenylase geranyl diphosphate:olivetolate geranyltransferase encodes the wild type or a mutant enzyme with yeast-preferred codons. In others, DNA constructs that encode bacterial, e.g., Streptomyces prenyltransferases with relaxed substrate specificities are used (Kumano et al., 2008).

In some embodiments, the host cell comprises one or more additional exogenous polynucleotides selected from the three following exogenous polynucleotides: an exogenous polynucleotide that encodes a prenol and isoprenol kinase; an exogenous polynucleotide that encodes a kinase that produces dimethylallyl pyrophosphate and isopentenyl pyrophosphate when grown in the presence of exogenous prenol and isoprenol; and an exogenous polynucleotide that encodes a geranyl-pyrophosphate synthase.

In contrast to previously described methodologies for the recombinant DNA-based production of cannabinoids in yeast, some embodiments of the present invention are based on the high aqueous solubility of both prenol and isoprenol together with the ability to generate recombinant host cells that express at high levels, heterologous kinase enzymes that can phosphorylate these 5-carbon compounds to the diphosphate level, thereby trapping them, due to the charged diphosphate moieties, within the host cell.

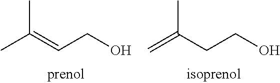

In some embodiments, the resulting diphosphates are then condensed to form geranyl-diphosphate (or pyrophosphate) through the action of either endogenous or heterologously expressed geranyl-pyrophosphate synthase (GPP synthase). This is then available for condensation with a 2-alkyl-4,6-dihydroxybenzoic acid through the action of a wild type or preferably a more active mutant aromatic prenyltransferase enzyme to form cannabigerolic acid or a cannabigerolic acid analog.

In other embodiments, geraniol itself is converted, through the actions of heterologously expressed kinase enzymes to form geranyl-pyrophosphate, which is then coupled with olivetolic acid or an olivetolic acid analog (e.g., 2-alkyl-4,6-dihydroxybenzoic acid), through the action of a wild-type prenyltransferase or a mutant prenyltransferase enzyme, to form cannabigerolic acid or a cannabigerolic acid analog.

In some embodiments, host cells are further modified to express a CBDA synthase (EC 1.21.3.8), a THCA synthase, or CBCA synthase as further described below.

### Engineering the Host Cell

Polynucleotides can be introduced into host cells using any methodology. In some embodiments, exogenous polynucleotides encoding two or more enzymes, e.g., two of: an acyl-CoA synthetase, such as revS or CsAAE3, or a transmembrane domain-deleted CsAAE1; a Type I or Type III polyketide synthase, such as MicC, Ben A, or multimeric BenA-BenB-BenC PKS; wherein when the PKS is MicC, a MicA polypeptide, and when the PKS is BenA, a BenQ polypeptide; and a 2-alkyl-4,6-dihydroxybenzoic acid cyclase (e.g., olivetolic acid cyclase) as described herein are present in the same expression construct, e.g., an autonomously replicating expression vector. In some embodiments, two or more of the enzymes are expressed as components of a multicistronic RNA in which expression is driven by the same promoter. Thus, for example, in some embodiments, an exogenous polynucleotide encoding a MicC polypeptide and an exogenous polynucleotide encoding an acylCoA synthetase, a 2-alkyl-4,6-dihydroxybenzoic acid cyclase, or a MicA polypeptide may be contained in an expression construct driven by the same promoter. In another example, in some embodiments, an exogenous polynucleotide encoding a BenA polypeptide and an exogenous polynucleotide encoding an acylCoA synthetase, a 2-alkyl-4,6-dihydroxybenzoic acid cyclase, or a BenQ polypeptide may be contained in an expression construct driven by the same promoter. In some embodiments, an expression vector, e.g., an autonomously replicating vector, may comprise two exogenous polynucleotides for generating a cannabinoid separated by an internal ribosome entry site (IRES) such that expression is driven by the same promoter to generate a discistronic mRNA. In some embodiments, the promoter is an alcohol dehydrogenase-2 promoter. In some embodiments, exogenous polynucleotides are present in the same expression construct, e.g., an autonomously replicating expression vector, and are operably linked to separate promoters. In some embodiments, exogenous polynucleotides are present in two or more expression constructs, e.g., autonomously replicating expression vectors. In some embodiments, the autonomously replicating expression vector is a yeast artificial chromosome. In some embodiments, one or more of the exogenous polynucleotides are integrated into the host genome. In some embodiments, multiple exogenous polynucleotides are introduced into the host cell by retrotransposon integration.

In some embodiments, a cannabinoid compound is produced using olivetol (5-pentyl-1,3-diol) or divarinol (5-propyl-1,3-diol) that is produced by genetically modified host cells as described herein, e.g., genetically modified to express BenA-BenB-BenC and the olivetol or divarinol can be modified chemically, e.g. to generate CBC and cannabinol (CBN) cor the propyl-derivatives CBCV and cannabinovarin (CBNV) as described by Crombie et al., Journal of the Chemical Society C: Organic, 796-804, 1971; Capriolglio et al., Org. Lett 21:6122-6125, 2019).

In some embodiments, a cannabinoid compound is produced using olivetolic acid or olivetolic acid analog that is expressed within the host cell, e.g., as described in the preceding paragraph, and the host cell is further modified to express a prenyltransferase, prenol and isoprenol kinase; a kinase to produce dimethylallyl pyrophosphate and isopentenyl pyrophosphate when grown in the presence of exogenous prenol and isoprenol; or a polynucleotide that encodes a geranyl-pyrophosphate synthase as described herein. Such polynucleotides may be contained in the same or separate expression vectors as described in the preceding paragraph.

Examples of prenyltransferases include, but are not limited to, geranylpyrophosphate:olivetolate geranyltransferase (GOT; EC 2.5.1.102) as described by Fellermeier & Zenk (FEBS Letters 427:283-285; 1998), as well as Cannabis sativa prenyltransferases described in WO 2018/200888 and WO 2019/071000. Streptomyces prenyltransferases including NphB, as described by Kumano et al. (Bioorg Med Chem. 16(17): 8117-8126; 2008), can also be used in accordance with the invention. In some embodiments, the prenyltransferase is fnq26: Flaviolin linalyltransferase from Streptomyces cinnamonensis. In some embodiments, a host cell genetically modified to express the prenyltransferase may be a modified host cell as described below.

In some embodiments, the modified recombinant host cell further comprises an exogenous polynucleotide that encodes a cannabinoid synthase enzyme that catalyzes conversion of a first cannabinoid compound intermediate produced in the host cell to form a second cannabinoid compound.

### Host Cells

In some embodiments, the host cell is a yeast or a filamentous fungus host cell such as an Aspergillus host cell. Genera of yeast that can be employed as host cells include, but are not limited to, cells of Saccharomyces, Schizosaccharomyces, Candida, Hansenula, Pichia, Kluyveromyces, Yarrowia and Phaffia. Suitable yeast species include, but are not limited to, Saccharomyces cerevisiae, Schizosaccharomyces pombe, Candida albicans, Hansenula polymorpha, Pichia pastoris, P. canadensis, Kluyveromyces marxianus, Kluyveromyces lactis, Phaffia rhodozyma and, Yarrowia lipolytica. Filamentous fungal genera that can be employed as host cells include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Mycehophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma. Illustrative species of filamentous fungal species include Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Mycehophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

In some embodiments, the host cell is selected from the group consisting of Saccharomyces cerevisiae, Kluyveromyces lactis, Kluyveromyces marxianus, Pichia pastoris, Yarrowia lipolytica, Hansenula polymorpha and Aspergillus.

In some embodiments, the yeast strain is a modified industrial ethanol producing strain and/or is strain “Super alcohol active dry yeast” (Angel Yeast Co., Ltd. Yichang, Hubei 443003, P.R. China). Such strains are modified by curing to cir⁰ and have selectable markers (e.g. URA3 and LEU2) integrated into the genome. Additional yeast strains that can be used include InvSc1 (MATa his3Δ1 leu2 trp1-289 ura3-52/MATαhis3Δ1 leu2 trp1-289 ura3-5) (Invitrogen), or the protease deficient strain BJ2168 (ATCC 208277 MATa prc1-407 prb1-1122 pep4-3 leu2 trp1 ura3-52 gal2).

In the above embodiments, the genes may be encoded by chemically synthesized genes, with yeast codon optimization, that encode a wild type or mutant enzyme from C. sativa, Arabidopsis thaliana or Pseudomonas spp.

Promoters used for driving transcription of genes in S. cerevisiae and other yeasts are well known in the art and include DNA elements that are regulated by glucose concentration in the growth media, such as the alcohol dehydrogenase-2 (ADH2) promoter. Other regulated promoters or inducible promoters, such as those that drive expression of the GAL1, MET25 and CUP1 genes, are used when conditional expression is required. GAL1 and CUP1 are induced by galactose and copper, respectively, whereas MET25 is induced by the absence of methionine.

In some embodiments, one or more of the exogenous polynucleotides is operably linked to a glucose regulated promoter. In some embodiments, expression of one or more of the exogenous polynucleotides is driven by an alcohol dehydrogenase-2 promoter.

Other promoters drive strongly transcription in a constitutive manner. Such promoters include, without limitation, the control elements for highly expressed yeast glycolytic enzymes, such as glyceraldehyde-3-phosphate dehydrogenase (GPD), phosphoglycerate kinase (PGK), pyruvate kinase (PYK), triose phosphate isomerase (TPI), enolase (ENO2), and alcohol dehydrogenase-1 (ADH1). Other strong constitutive promoters that may be used are those from the S. cerevisiae transcription elongation factor EF-1 alpha genes (TEF1 and TEF2) (Partow et al., Yeast. 2010, (11):955-64; Peng et al., Microb Cell Fact. 2015, (14):91-102) and the high-affinity glucose transporter (HXT7) and chaperonin (SSA1) promoters that function well under conditions of low glucose following the S. cerevisiae diauxic shift (Peng et al., Microb Cell Fact. 2015, (14):91-102).

In other embodiments, the host cells can increase cannabinoid production by increasing precursor pools and the like. Heterologous natural or chemically synthesized genes for enzymes such as malonyl-CoA synthase, with malonate feeding (Mutka et al., FEMS Yeast Res. 2006), and acetyl-CoA carboxylases 1 and 2 up-regulate the important malonyl-CoA for PKS biosynthesis. Similarly, acetyl-CoA synthases-1 and -2, and other gene products in the mevalonate pathway, e.g., acetoacetyl-CoA thiolase or the NphT7 gene product from Streptomyces sp. (Okamura et al., Proc Natl Acad Sci USA. 2010), HMG-CoA synthase, mevalonate kinase, phosphomevalonate kinase, mevalonate diphosphate decarboxylase, isopentenyl diphosphate:dimethylallyl diphosphate isomerase, HMG-CoA reductase, mutant farnesyl-pyrophosphate synthase (ERG20; Zhao et al., 2016) from Saccharomyces or other eukaryotic species may also be introduced on high-level expression plasmid vectors or through genomic integration using methods well known to those skilled in the art. Such methods may involve CRISPR Cas-9 technology, yeast artificial chromosomes (YACs) or the use of retrotransposons. Alternatively, if natural to the host organism, such genes may be up-regulated by genetic element integration methods known to those skilled in the art.

In yet other aspects, similar engineering may be employed to reduce the production of natural products, e.g., ethanol that utilize carbon sources that lead to reduced utilisation of that carbon source for cannabinoid production. Such genes may be completely “knocked out” of the genome by deletion, or may be reduced in activity through reduction of promoter strength or the like. Such genes include those for the enzymes ADH1 and/or ADH6. Other gene “knockouts” include genes involved in the ergosterol pathway, such as ERG9 and the two most prominent aromatic decarboxylase genes of yeast, PAD1 and FDC1.

Further embodiments include genes for accessory enzymes aimed at assisting in the production of the final product cannabinoids. One such enzyme, catalase, is able to neutralize hydrogen peroxide produced by certain enzymes involved in the oxido-cyclization of CBGA and analogs, such as cannabidiolic acid synthase (Taura et al., 2007), Δ⁹-tetrahydrocannabinolic acid synthase (Sirikantaramas et al., 2004) and cannabichromenic acid synthase (Morimoto et al., 1998).

In further embodiments, the engineered host cells contain up-regulated or down-regulated endogenous or heterologous genes to optimize, for example, the precursor pools for cannabinoid biosynthesis. Additional, further heterologous gene products may be expressed to give “accessory” functions within the cell. For example, overexpressed catalase may be expressed in order to neutralize hydrogen peroxide formed in the oxido-cyclization step to important acidic cannabinoids such as CBDA, Δ⁹-THCA and CBCA. “Accessory” genes and their expressed products may be provided through integration into the yeast genome through techniques well known in the art, or may be expressed from plasmids (also known as yeast expression vectors), yeast artificial chromosomes (YACs) or yeast transposons.

In some embodiments, host cells, e.g., yeast strains, transformed or genomically integrated with plasmids or vectors containing each of the above genes are transformed together with another expression system for the conversion of CBGA or a CBGA analog to a second acidic cannabinoid, as further explained below. In some such embodiments, the expression system is on the same vector or on a separate vector, or is integrated into the host cell genome.

The cannabinoid-producing engineered cells of the invention may be made by transforming a host cell, either through genomic integration or using episomal plasmids (also referred to as expression vectors, or simply vectors) with at least one nucleotide sequence encoding enzymes involved in the engineered metabolic pathways. As used herein the term “nucleotide sequence”, “nucleic acid sequence” and “genetic construct” are used interchangeably and mean a polymer of RNA or DNA, single- or double-stranded, optionally containing synthetic, non-natural or altered nucleotide bases. A nucleotide sequence may comprise one or more segments of cDNA, genomic DNA, synthetic DNA, or RNA. In some embodiments, the nucleotide sequence is codon-optimized to reflect the typical codon usage of the host cell without altering the polypeptide encoded by the nucleotide sequence. In certain embodiments, the term “codon optimization” or “codon-optimized” refers to modifying the codon content of a nucleic acid sequence without modifying the sequence of the polypeptide encoded by the nucleic acid to enhance expression in a particular host cell. In certain embodiments, the term is meant to encompass modifying the codon content of a nucleic acid sequence as a means to control the level of expression of a polypeptide (e.g., either increase or decrease the level of expression). Accordingly, described are nucleic sequences encoding the enzymes involved in the engineered metabolic pathways. In some embodiments, a metabolically engineered cell may express one or more polypeptide having an enzymatic activity necessary to perform the steps described below. In some embodiments, the nucleotide sequences are synthesized and codon-optimized for expression in yeast according to methods described in U.S. Pat. No. 7,561,972.

For example a particular cell may comprises one, two, three, four, five or more than five nucleic acid sequences, each one encoding the polypeptide(s) necessary to produce a cannabinoid compound, or cannabinoid compound intermediate described herein. Alternatively, a single nucleic acid molecule can encode one, or more than one, polypeptide. For example, a single nucleic acid molecule can contain nucleic acid sequences that encode two, three, four or even five different polypeptides. Nucleic acid sequences useful for the invention described herein may be obtained from a variety of sources such as, for example, amplification of cDNA sequences, DNA libraries, de novo synthesis, excision of genomic segment. The sequences obtained from such sources may then be modified using standard molecular biology and/or recombinant DNA technology to produce nucleic sequences having desired modifications. Exemplary methods for modification of nucleic acid sequences include, for example, site directed mutagenesis, PCR mutagenesis, deletion, insertion, substitution, swapping portions of the sequence using restriction enzymes, optionally in combination with ligation, homologous recombination, site specific recombination or various combination thereof. In other embodiments, the nucleic acid sequences may be a synthetic nucleic acid sequence. Synthetic polynucleotide sequences may be produced using a variety of methods described in U.S. Pat. No. 7,323,320, as well as U.S. Pat. Appl. Pub. Nos. 2006/0160138 and 2007/0269870. Methods of transformation of yeast cells are well known in the art.

### IV. Methods for Cannabinoid Production

### Fermentation Conditions

Cannabinoid production according to the methods provided herein generally includes the culturing of host cells (e.g., yeast or filamentous fungi) that have been engineered to contain the expression systems described above. In some embodiments, the carbon sources for yeast growth are sugars such as glucose, dextrose, xylose, or other sustainable feedstock sugars such as those derived from cellulosic sources, for example. In other embodiments, the carbon sources used may be methanol, glycerol, ethanol or acetate. In some embodiments, feedstock compositions are refined by experimentation to provide for optimal yeast growth and final cannabinoid production levels, as measured using analytical techniques such as HPLC. In such embodiments, methods include utilization of glucose/ethanol or glucose/acetate mixtures wherein the molar ratio of glucose to the 2-carbon source (ethanol or acetate) is between the ranges of 50/50, 60/40, 80/20, or 90/10. Feeding may be optimized to both induce glucose-regulated promoters and to maximize the production of acetyl-CoA and malonyl-CoA precursors in the production strain.

Fermentation methods may be adapted to a particular yeast strain due to differences in their carbon utilization pathway or mode of expression control. For example, a Saccharomyces yeast fermentation may require a single glucose feed, complex nitrogen source (e.g., casein hydrolysates), and multiple vitamin supplementation. This is in contrast to the methylotrophic yeast Pichia pastoris which may require glycerol, methanol, and trace mineral feeds, but only simple ammonium (nitrogen) salts, for optimal growth and expression. See, e.g., Elliott et al. J. Protein Chem. (1990) 9:95 104, U.S. Pat. No. 5,324,639 and Fieschko et al. Biotechnol. Bioeng. (1987) 29:1113 1121. Culture media may contain components such as yeast extract, peptone, and the like. The microorganisms can be cultured in conventional fermentation modes, which include, but are not limited to, batch, fed-batch, and continuous flow.

In some embodiments, the rate of glucose addition to the fermenter is controlled such that the rate of glucose addition is approximately equal to the rate of glucose consumption by the yeast; under such conditions, the amount of glucose or ethanol does not accumulate appreciably. The rate of glucose addition in such instances can depend on factors including, but not limited to, the particular yeast strain, the fermentation temperature, and the physical dimensions of the fermentation apparatus.

For the MPF procedure, in batch mode, the precursors olivetolic acid (or an olivetolic acid analog such as another 2-alkyl-4,6-dihydroxybenzoic acid), olivetol (or an olivetol analog such as another 5-alkylbenzene-1,3-diol), prenol, isoprenol or geraniol may be present in concentrations of between 0.1 and 50 grams/L (e.g., between 1 and 10 g/L). In fed-batch mode, the precursors may be fed slowly into the fermentation over between 2 and 20 hours, such that a final addition of between 1 and 100 grams/L (e.g., between 1 and 10 grams/L, or between 10 and 100 grams/L) of each requisite precursor occurs.

Similarly, carboxylic acid starting materials such as hexanoic acid, butanoic acid, pentanoic acid, and the like may be present in concentrations of between 0.1 and 50 grams/L (e.g., between 1 and 10 g/L). In fed-batch mode, the carboxylic acid may be fed slowly into the fermentation over between 2 and 20 hours, such that a final addition of between 1 and 100 grams/L (e.g., between 1 and 10 grams/L, or between 10 and 100 grams/L) of the carboxylic acid occurs.

Culture conditions such as expression time, temperature, and pH can be controlled so as to afford target cannabinoid intermediates (e.g., olivetolic acid) and/or target cannabinoid products (e.g., CBGA, CBG) in high yield. Host cells are generally cultured in the presence of starting materials, such as hexanoic acid, prenol, isoprenol, or the like, for periods of time ranging from a few hours to a day or longer (e.g., 24 hours, 30 hours, 36 hours, or 48 hours) at temperatures ranging from about 20° C. to about 40° C. depending on the particular host cells employed. For example, S. cerevisiae may be cultured at 25-32° C. for 24-40 hours (e.g., 30 hours). The pH of culture medium can be maintained at a particular level via the addition of acids, bases, and/or buffering agents. In certain embodiments, culturing yeast at a pH of 6 or higher can reduce the production of unwanted side products such as olivetol. In some embodiments, the pH of the yeast culture ranges from about 6 to about 8. In some embodiments, the pH of the yeast culture is about 6.5. In some embodiments, the pH of the yeast culture is about 7. In some embodiments, the pH of the yeast culture is about 8.

In some embodiments, a recombinant yeast cell is genetically modified such that it produces, when cultured in vivo in a suitable precursor-containing media as described above, the cannabinoid product of interest or an intermediate at a level of at least about 0.1 g/L, at least about 0.5 g/L, at least about 0.75 g/L, at least about 1 g/L, at least about 1.5 g/L, at least about 2 g/L, at least about 2.5 g/L, at least about 3 g/L, at least about 3.5 g/L, at least about 4 g/L, at least about 4.5 g/L, at least about 5 g/L, at least about 5.5 g/L, at least about 6 g/L, at least about 7 g/L, at least about 8 g/L, at least about 9 g/L, or at least 10 g/L. In some embodiments, a recombinant yeast cell is genetically modified such that it produces, when cultured in vivo in a suitable medium, the cannabinoid product of interest or an intermediate at a level of at least about 20 g/L, at least about 30 g/L, at least about 50 g/L, or at least about 80 g/L.

Cannabinoid production may be carried out in any vessel that permits cell growth and/or incubation. For example, a reaction mixture may be a bioreactor, a cell culture flask or plate, a multiwell plate (e.g., a 96, 384, 1056 well microtiter plates, etc.), a culture flask, a fermenter, or other vessel for cell growth or incubation. Biologically produced products of interest may be isolated from the fermentation medium or cell extract using methods known in the art. For example, solids or cell debris may be removed by centrifugation or filtration. Products of interest may be isolated, for example, by distillation, liquid-liquid extraction, membrane evaporation, adsorption, or other methods.

### Conversion of Cannabinoid Starting Materials to Cannabinoid Products

Also provided herein are methods for producing cannabinoid products. In some embodiments, the methods include expressing a cannabinoid starting material (e.g., a 5-alkyl-benzene-1,3-diol, a 2-alkyl-4,6-dihydroxybenzoic acids, or a combination thereof), in a yeast cell, wherein the yeast cell is genetically modified to express the cannabinoid starting material, isolating the yeast cell, and converting the cannabinoid starting material to the cannabinoid product in the isolated yeast cell. As used herein with respect to producing cannabinoid products using a Type I or Type II PKS, the term “cannabinoid precursor product” may also be used to refer to a cannabinoid starting material 5-alkyl-benzene-1,3-diol, or a 2-alkyl-4,6-dihydroxybenzoic acids, or a combination thereof. In some embodiments, such a cannabinoid precursor product is olivetol, olivetolic acid, divarinol, or divarinic acid. The cannabinoid starting material can be an acidic cannabinoid, a neutral cannabinoid, or a cannabinoid precursor such as olivetolic acid (or another 2-alkyl-4,6-dihydroxybenzoic acid) or olivetol (or another 5-alkylbenzene-1,3-diol). Converting the cannabinoid starting material can be conducted using the procedures described herein (e.g., chemical or enzymatic geranylation, thermal or enzymatic decarboxylation, etc.) or can be modified according to the identity of the particular cannabinoid starting material or the particular cannabinoid product. The cannabinoid starting material can be expressed, for example, using any of the expression systems described above. Isolating the yeast cells can optionally include: collecting yeast cells from culture media by centrifugation, filtration, or other means; washing yeast cells to remove culture media or other components; removing at least a portion of liquid (e.g., culture media) from the cells; and/or drying the cells (e.g., by lyophilization or other means). Isolated yeast cells can be directly subjected to reaction conditions for forming the cannabinoid products. For example, yeast cells can be combined directly with solvents and other reagents as described below.

In some embodiments, a yeast cell genetically modified to express a cannabinoid starting material as described herein produces olivetol or divarinol, which can be chemically modified to produce a cannabinoid.

In some embodiments, the methods include culturing modified recombinant host cells containing an expression system as described above under conditions in which a 2-alkyl-4,6-dihydroxybenzoic acid or 5-alkylbenzene-1,3-diol is produced, and converting the 2-alkyl-4,6-dihydroxybenzoic acid or 5-alkylbenzene-1,3-diol to the cannabinoid product. In some embodiments, the methods include culturing modified recombinant host cells containing an expression system as described above under conditions in which olivetolic acid or olivetol is produced, and converting the olivetolic acid or olivetol to the cannabinoid product.

In some embodiments, the converting step is conducted in vitro. For example, the converting step can include forming a reaction mixture comprising (i) a 2-alkyl-4,6-dihydroxybenzoic acid (e.g., olivetolic acid) or a 5-alkylbenzene-1,3-diol (e.g., olivetol), geraniol, (ii) an activated geraniol (e.g., geranyl bromide, geranyl chloride, geranyl tosylate, geranyl mesylate, or the like), or citral, and (iii) an organic solvent under conditions sufficient to produce an acidic cannabinoid (e.g., cannabigerolic acid, CBGA, or cannabichromenic acid, CBCA) or a neutral cannabinoid (e.g., cannabigerol, CBG, or cannabichromene, CBC). The method can be employed to convert olivetolic acid analogs to the corresponding acidic cannabinoids, or to convert olivetol analogs to the corresponding neutral cannabinoids.

Any suitable organic solvent can be used in the methods of the invention. Suitable solvents include, but are not limited to, toluene, methylene chloride, ethyl acetate, acetonitrile, tetrahydrofuran, benzene, ethylbenzene, xylenes (i.e., m-xylene, o-xylene, p-xylene, or any combination thereof), chloroform, diethyl ether, dimethyl formamide, dimethyl sulfoxide, petroleum ether, and mixtures thereof. In some embodiments, the organic solvent is toluene, benzene, ethylbenzene, xylenes, or a mixture thereof. In some embodiments, the organic solvent is toluene. Aqueous organic solvent mixtures (i.e., a mixture of water and a water-miscible organic solvent such as tetrahydrofuran or dimethyl formamide) can also be employed. In general, the ratio of the solvent to the 2-alkyl-4,6-dihydroxybenzoic acid or 5-alkylbenzene-1,3-diol ranges from about 1:1 to about 1000:1 by weight. The ratio of the solvent to the 2-alkyl-4,6-dihydroxybenzoic acid or 5-alkylbenzene-1,3-diol can be, for example, about 100:1 by weight, or about 10:1 by weight, or about 5:1 weight. In certain embodiments, the 2-alkyl-4,6-dihydroxybenzoic acid or 5-alkylbenzene-1,3-diol is present in a yeast mixture (e.g., dried yeast cells, or a wet yeast cell pellet collected from culture). In some such embodiments, the reaction mixture comprises the host cell (e.g., dried yeast cells). The ratio of solvent to yeast mixture (e.g., dried yeast cells) can range from about 1:1 to about 1000:1 by weight. The ratio of the solvent to the yeast mixture can be, for example, about 100:1 by weight, or about 10:1 by weight, or about 5:1 by weight, or about 2:1 by weight.

Any suitable amount of geraniol, activated geraniol, or citral can be used in the conversion step. In general, the reaction mixture contains at least one molar equivalent of geraniol, activated geraniol, or citral with respect to the 2-alkyl-4,6-dihydroxybenzoic acid or 5-alkylbenzene-1,3-diol. The reaction mixture can contain, for example, from about 1 molar equivalent to about 10 molar equivalents of geraniol, activated geraniol, or citral, with respect to the 2-alkyl-4,6-dihydroxybenzoic acid or 5-alkylbenzene-1,3-diol (e.g., about 1.1 molar equivalents, or about 1.2 molar equivalents, or about 2 molar equivalents).

In some embodiments, the reaction mixture further comprises an acid. Any suitable acid can be used in the conversion step. Examples of suitable acids include, but are not limited to, hydrochloric acid, sulfuric acid, nitric acid, formic acid, acetic acid, trifluoroacetic acid, p-toluenesulfonic acid, methanesulfonic acid, and trifluoromethane sulfonic acid. In some embodiments, the acid is a sulfonic acid. In some embodiments, the acid is p-toluenesulfonic acid. Any suitable amount of the acid can be used in the conversion step. In general, the reaction mixture contains from about 0.01 molar equivalents of the acid (e.g., p-toluenesulfonic acid) to about 10 molar equivalents of the acid with respect to the 2-alkyl-4,6-dihydroxybenzoic acid or 5-alkylbenzene-1,3-diol (e.g., about 0.01 molar equivalents, or about 0.1 molar equivalents).

In some embodiments, the reaction mixture further comprises an amine. Examples of suitable amines include, but are not limited to, N,N-diisopropylethylamine, trimethylamine, pyridine, and diamines (e.g., a 1,2-diamine). Examples of suitable diamines include, but are not limited to, ethylene diamine, N,N-dimethylethylenediamine, N,N-diethylethylenediamine, N,N′-dimethylethylenediamine, N,N′-diphenylethylenediamine, N,N′-dibenzylethylenediamine, and N,N′-bis(2-hydroxyethyl)ethylenediamine. In some embodiments, the reaction mixture includes citral and N,N-dimethylethylenediamine. Any suitable amount of the amine can be used in the conversion step. In general, the reaction mixture contains from about 0.01 molar equivalents of the amine (e.g., N,N-dimethylethylenediamine) to about 10 molar equivalents of the amine with respect to the 2-alkyl-4,6-dihydroxybenzoic acid or 5-alkylbenzene-1,3-diol (e.g., about 0.01 molar equivalents, or about 0.25 molar equivalents, or about 0.1 molar equivalents, or about 1 molar equivalent).

The converting step can be conducted at any suitable temperature. Typically, the conversion step is conducted at temperatures ranging from about 20° C. to about 200° C., e.g., from about 25° C. to about 100° C., or from about 25° C. to about 80° C., or from about 25° C. to about 70° C. The conversion step is conducted for a period of time sufficient to convert the 2-alkyl-4,6-dihydroxybenzoic acid or 5-alkylbenzene-1,3-diol to the cannabinoid product (e.g., to convert olivetolic acid to CBGA, or to convert olivetol to CBG). Depending on factors such as the particular acid employed, the particular solvent employed, and the state of the 2-alkyl-4,6-dihydroxybenzoic acid or 5-alkylbenzene-1,3-diol (e.g., present in a yeast mixture), the conversion time will range from a few minutes to several hours. In some embodiments, the reaction mixture will be maintained at a temperature ranging from about 25° C. to about 100° C. (e.g., about 60° C.) for a period of time ranging from about 5 minutes to about 360 minutes. In some embodiments, the reaction mixture is maintained at or around 60° C. for 60 minutes or less (e.g., about 55 minutes, or about 30 minutes, or about 15 minutes, or about 10 minutes).

In some embodiments, an acidic cannabinoid such as CBGA is the cannabinoid product. In some embodiments, the method further includes converting the acidic cannabinoid, e.g., CBGA, to the cannabinoid product. The final cannabinoid product can be a neutral cannabinoid or another acidic cannabinoid. In some embodiments, conversion of an intermediate compound such as CBGA to another cannabinoid is carried out via physical or chemical processes such as heating, auto-oxidation or UV light treatment. For example, the methods can include the decarboxylation of acidic cannabinoid, either within the engineered yeast cells or following their full or partial purification through the action of heat or through the action of a wild-type or mutant decarboxylase enzyme contacting the cannabinoid acid in vivo or in vitro. Decarboxylation of the acidic cannabinoids provides corresponding neutral cannabinoids; decarboxylation of CBGA, for example, provides CBG.

In some embodiments, UV light treatment, heating, oxidation, or other reaction conditions are employed such that a first intermediate recombinant DNA-derived cannabinoid product is retained within the yeast cells and is then converted to a second valuable cannabinoid product that is isolated and purified at commercial scale.

Additional chemical transformations may be performed on the cannabinoids formed to make fully non-natural analogs such as esters, ethers and halogenated derivatives, either for use as pro-drugs, or more active or bioavailable drug substances. In some embodiments, this chemistry may be performed on whole yeast cells that harbor the biosynthetic cannabinoid substrates in order to avoid unnecessary purification steps prior to formation of the desired final product.

In still other embodiments, described is a method for conversion of a first intermediate cannabinoid to a second cannabinoid through the action of a wild type or a mutant cannabinoid or cannabinoid acid synthase, either within the same engineered host cell or through co-culturing with two or more recombinant host cell strains, e.g., yeast strains.

As explained above, in some embodiments, host cells, e.g., yeast strains, transformed or genomically integrated with plasmids or vectors containing each of the above genes are transformed together with another expression system for the conversion of CBGA or a CBGA analog to a second acidic cannabinoid. In some such embodiments, the expression system is on the same vector or on a separate vector, or is integrated into the host cell genome. In other embodiments, the expression system for the conversion activity encodes one of the C. sativa enzymes THCA synthase, CBDA synthase or CBCA synthase. In some embodiments, the synthase is a homolog from hops, e.g., a CBDA synthase homolog from hops.

In some embodiments, an acidic cannabinoid, e.g., CBGA or CBDA, may be decarboxylated to form a neutral cannabinoid compound, e.g., CBG or CBD, using a decarboxylase, e.g., Aspergillus nidulans orsB decarboxylase. Alternatively, an acidic cannabinoid can be decarboxylated by maintaining the acidic cannabinoid at an elevated temperature (e.g., around 40° C., 50° C., or 100° C.) for periods of time ranging from a few minutes to several hours.

The invention illustratively described herein suitably may be practiced in the absence of any element or elements, limitation or limitations that are not specifically disclosed herein. Thus, for example, in each instance herein any of the terms “comprising”, “consisting essentially of” and “consisting of” may be replaced with either of the other two terms. Thus, for example, some embodiments may encompass a host cell “comprising” a number of components, other embodiments would encompass a host cell “consisting essentially of” the same components, and still other embodiments would encompass a host cell “consisting of” the same components. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art. All patents, patent applications, and literature references cited in the present specification are hereby incorporated by reference in their entirety.

### V. Examples

### Example 1. Production of 2-hydroxy-6-pentylbenzoic acid and 2,4-dihydroxy-6-pentylbenzoic Acid (Olivetolic Acid) in S. cerevisiae Using Micacocidin Gene Cluster Genes

The S. cerevisiae ADH2 promoter is chemically synthesized and fused to a synthetic gene for a mutated C. sativa acyl-activating enzyme-1 in which the transmembrane domain coding sequences (amino acids 245 to 267) were deleted (CsAAE1ΔTM). An S. cerevisiae ADH2 terminator sequence is also fused to the gene sequence immediately subsequent to the synthetic stop codons. The expression cassette is cloned into a yeast expression vector containing the URA3 selectable marker. Similarly, synthetic genes for the acyl-activating enzymes CsAAE3 (from C. sativa) and revS (a middle chain fatty acyl-CoA ligase from Streptomyces sp. SN-593) are cloned into separate URA3 vectors for separate evaluation, e.g., in parallel. Each URA3-based vector is transformed into competent Saccharomyces cerevisiae InvSc1 (MAT1a his3D1 leu2 trp1-289 ura3-52MAT alpha his3D1 leu2 trp1-289 ura3-52) cells (Invitrogen) that are previously transformed with selectable marker LEU2-based vectors containing Streptomyces micA, micC genes and a truncated micC gene fused, via the S. cerevisiae p150 internal ribosome entry site (IRES) and a human ubiquitin gene, to a number of PPTase genes, including sfp and NpgA for evaluation. Variants of the micC gene product include truncated (amino acids 1-2700) proteins and ketoreductase domain mutated enzymes.

Transformed cells are plated on minimal agar plates (6.7 g/L yeast nitrogen base without amino acids or ammonium sulfate (DIFCO), 20 g/L glucose, 20 g/L agar) containing amino acids for selection based on uracil and leucine prototrophy. Transformants are picked and grown for 24 hours in uracil- and leucine-deficient minimal medium. Plasmid DNA was isolated from the transformants and analyzed by restriction digestion analysis to confirm identity.

A successful transformant for each strain is used to inoculate 2 mL of uracil- and leucine-deficient minimal medium that was grown overnight at 30° C. in an orbital shaker. A 500-μL aliquot of this culture is used to inoculate 50 mL of the same media and the culture is grown at 30° C. in a shaker for 24 h. The culture is similarly inoculated into 300 mL of the same media and, after overnight growth, is transferred into an oxygen-, feed-, and agitation-controlled 7.5-liter fermenter (Eppendorf) containing 1.7 L 2×YEPD medium (Wobbe, in Current Protocols in Molecular Biology, Supplement 34:13.0.1-13.13.9 (Wiley, 1996)) (10 g/L yeast extract, 20 g/L peptone, 20 g/L glucose).

After approximately 16 hours post inoculation, following consumption of all residual glucose, the culture is fed with 2X YEP that contained 14.3% glucose, 3.5% sodium acetate and 1 gram of hexanoic acid or a hexanoic acid analog, through to an elapsed fermentation time of 72 hours.

Cells are collected by centrifugation of 500-μL aliquots of the culture taken after 24, 48, and 72 hours of growth and lysed by boiling in 50 μL of 2×SDS gel loading buffer for about 2 minutes. The cell lysates are analyzed by loading onto 12% SDS-PAGE gels. Bands corresponding to the expected sizes of the encoded enzymes were observed.

For further quantitation and for embodiments in which analogs are generated, analog verification, cells are separated from the media by centrifugation, the media is acidified with glacial acetic acid, and the products are extracted using ethyl acetate. The products are further purified by column chromatography, or using Sep-Pak C18 cartridges with acetonitrile/formic acid elution, and subjected to NMR and mass spectroscopy analysis.

High levels (multi-100 mg/L) of the analogs are biosynthesized with the relative yield distribution using the various acyl-activating enzymes being in the order: revS>CsAAE3>CsAAE1≅CsAAE1ΔTM. Product distribution of olivetolic acid to olivetol analog varies with the actual length of the mutated cyclase used, with the AtHS1 cyclase giving essentially all olivetol (5-pentylbenzene-1,3-diol).

### Example 2. Production of 2,4-dihydroxy-6-pentylbenzoic Acid (Olivetolic Acid) and 2,4-dihydroxy-6-propylbenzoic Acid (Divarinic Acid) and their Analogs in S. cerevisiae Using Benastatin Gene Cluster Genes

The S. cerevisiae ADH2 promoter was chemically synthesized and fused to a synthetic gene for BenA that was designed using yeast-preferred codons. An S. cerevisiae Alpha factor terminator sequence was also fused to the gene sequence immediately subsequent to the synthetic stop codons. Synthetic genes for benB under the control of the S. cerevisiae tef1 promoter and CYC terminator and the contiguous benC gene, under the control of the S. cerevisiae pyk1 promoter and ADH2 terminator were cloned into the pBM211U and pBM211L plasmids to form plasmids pBM248U and pBM248L that expressed BenA, BenB and BenC when transformed into S. cerevisiae. Each URA3- or LEU2-based vector was transformed into competent Saccharomyces cerevisiae yBM4 cells that were previously transformed with selectable marker URA3- or LEU2-based vectors containing the C. sativa olivetolic acid synthase/tetraketide synthase (OAS/TKS) gene fused, via the S. cerevisiae p150 internal ribosome entry site (IRES) and a human ubiquitin gene, to a synthetic gene encoding amino acids 1-147 of the benH gene.

Transformed cells were plated on minimal agar plates (6.7 g/L yeast nitrogen base without amino acids or ammonium sulfate (DIFCO), 20 g/L glucose, 20 g/L agar) containing amino acids for selection based on uracil and leucine prototrophy. Transformants were picked and grown for 24 hours in uracil- and leucine-deficient minimal medium. Plasmid DNA was isolated from the transformants and analyzed by restriction digestion analysis to confirm identity.

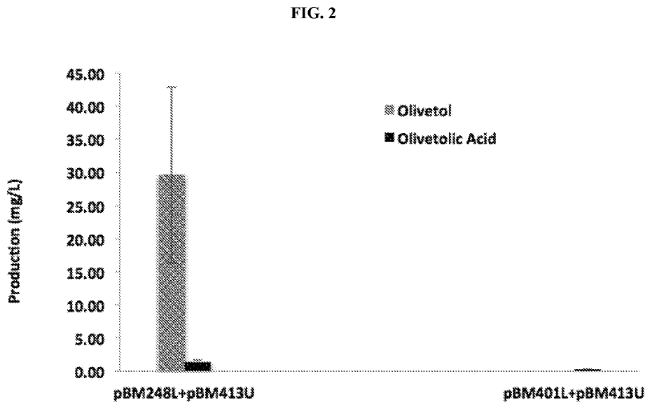

Strains expressing the BenABC and benH constructs, as described above, were grown in 4 mL of selective media at 30° C. for 24 h and then inoculated into 2×YEPD, giving a total of 40 mL of cell culture volume. After 30 h of growth at 30° C., hexanoic acid, butanoic acid or 5-fluoropentanoic acid were added to the cultures to give a total concentration of 2 mM, and the cultures were grown at 30° C. for a further 48 h. Olivetol and olivetolic acid, divarinol and divarinic acid, and the corresponding fluoro-analog production was monitored by HPLC. Yields of olivetol were around 30 mg/L, and yields of olivetolic acid were around 1 mg/L (FIG. 2). A successful transformant for each strain was used to inoculate 2 mL of uracil- and leucine-deficient minimal medium that was grown overnight at 30° C. in an orbital shaker. A 500-μL aliquot of this culture was used to inoculate 50 mL of the same media and the culture was grown at 30° C. in a shaker for 24 h. The culture was similarly inoculated into 300 mL of the same media and, after overnight growth, was transferred into an oxygen-, feed-, and agitation-controlled 7.5-liter fermenter (Eppendorf) containing 1.7 L 2×YEPD medium (Wobbe, in Current Protocols in Molecular Biology, Supplement 34:13.0.1-13.13.9 (Wiley, 1996)) (10 g/L yeast extract, 20 g/L peptone, 20 g/L glucose).

After approximately 16 hours post inoculation, following consumption of all residual glucose, the culture was fed with 1 L of 2×YEP that contained 14.3% glucose, 3.5% sodium acetate and 1 gram of hexanoic acid, through to an elapsed fermentation time of 72 hours.

Cells were collected by centrifugation of 500-μL aliquots of the culture taken after 24, 48, and 72 hours of growth and lysed by boiling in 50 μL of 2×SDS gel loading buffer for about 2 minutes. The cell lysates were analyzed by loading onto 12% SDS-PAGE gels. Bands corresponding to the expected sizes of the encoded enzymes were observed.

The results (FIG. 2) showed production of olivetol and olivetolic acid in a yeast strain expressing BenA, BenB and BenC genes on one plasmid, and BenH on a second plasmid (left), compared with a control expressing the C. sativa tetraketide synthase and BenH (right). Yeast cells expressing BenA only yielded no polyketide products in this experiment.

In this experiment, the results indicate that it was not necessary to modify the cells to express an acyl-CoA synthetase in order to generate olivetol and olivetolic acid.

### Example 3. Use of an Organic Phase Overlay to Reduce Toxicity of Starting Materials and Products

Hexanoic acid, and butanoic acid are fed individually to the yeast strains described above in Examples 1 and 2. Culturing of the cells proceeded as described in Example 2, except that at 30 h, 10% by volume of oleyl alcohol is added to the culture along with the aliphatic acid or an aliphatic acid analog. This procedure leads to increased levels of the desired products.

### Example 4. Production of CBGA, CBGVA and their Analogs Directly in S. cerevisiae

Hexanoic acid and butanoic acid, are fed individually to yeast strains grown as described above in Examples 1 and 2, except that the strains are previously modified by integrative transformation of genes involved in the up-regulation of the yeast mevalonate pathway such that they produce high levels of geranyl-diphosphate. The strains also harbor integrated genes that individually express various prenyltransferases for conversion of olivetolic and divarinic acids and their analogs to CBGA, CBGVA and their analogs. The resulting CBGA, CBGVA and their analogs are isolated from centrifuged yeast cells by solvent extraction using methanol, ethanol or ethyl acetate, and are characterized by mass spectrometry and NMR analysis.

### Example 5. Chemical Transformation of Olivetol/Olivetolic Acid Analogs to CBC/CBCA Analogs

CBCA and CBC analogs were prepared as follows: to a 0.5 mL dichloroethane solution of 35 mg (0.2 mmol) of (perdeuteropentyl)-olivetolic acid or (perdeuteropentyl)-olivetol was added 0.085 mL (approximately 2.5 equiv) of E/Z-citral followed by addition of 0.005 mL (25 mol %) of N,N-dimethylethylene diamine to initiate the reaction at 23° C. The reaction was monitored by quantitative RP-HPLC and after 18 h, no substrate remained. The reaction mixture was purified directly by a single injection on a Gilson preparative C18 RP-HPLC automated system using a steep linear gradient of water/MeOH/0.1% formic acid (25 mL/min). Fractions were monitored by UV (at 230 nm) and the appropriate fractions were combined, concentrated in vacuo, and re-concentrated in MeOH to remove residual water, to afford products in molar yields ranging from 65% to 73%. CBCA and CBC analogs were characterized by mass spectrometry and NMR analysis.

### Example 6. Chemical Transformation of Olivetolic and Divarinic Acids and their Analogs to CBGA, CBGVA and their Analogs

To a suspension of 20 mg of olivetolic acid, divarinic acid or their analogs in 0.25 mL of toluene is added 2.6 mg of p-toluenesulphonic acid and 18 μL of geraniol. The suspension is heated to 60° C. and monitored by reversed-phase HPLC (Kinetex 5 μm-XB, 50×4.6 mm, 100 A, linear gradient of 20% 50 mM ammonium formate/acetonitrile to 100% acetonitrile over 6 min. at 2.5 mL/min.). The corresponding CBGA, CBGVA and their analogs reach maximal yield after approximately 50 minutes, and are identified and characterized by mass spectrometry and NMR.

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

ILLUSTRATIVE SEQUENCES
Ralstonia solanacearum MicC amino acid
sequence. In typical embodiments, the
MicC amino acid sequence comprises a
Y1991A amino acid substitution (Y1991
is underlined in SEQ ID NO: 1)
SEQ ID NO: 1
MTTHALTERATLVDWIEHHARARPLAEALFFCGHGADDLRLGYGALSERV
RRCAAALQQRGAAGSTALILFPSGIDYVVALLACFYAGVTGVPVNLPGVS
RVRRVLPKLGDITRDCRPAVVLTHTAIERASGNDLRDFAAGHGLDILHLD
TLGGEAAAWVRPALTPESIAFLQYTSGSTGSPKGVVNRHGALLRNLQFLG
RLTRPQDRAPEDTAVASWLPLFHDLGLIMGILLPLAYGNRAVYMAPMAFV
ADPLRWLEIATAERATALPCPSFALRLCADEARRAAPARTAGIDLSSVQC
LMPAAEPVLPSQIEAFQAAFAAHGMRREAIRPAYGLAEATLLVSANVDDA
PPHRIDVETAPLEQGRAVVHPAAAPMPAAGRRRYVSNGREFDGQDVRIVD
PRTCATLPEGTVGEIWISGPCIAGGYWNKAELNREIFMAETPGAGDRRYL
RTGDMGFLHGGHLFVTGRLKDMMLFRGQCHYPNDTEATSGRAHAAAIPES
GAAFSIQAEDEAGERLVIVQEVRKQAGIDPRDIATAVRAAVAEGHALGVH
AVVLIRKGTLPRTTSGKVRRAAVREAWLAGTLQTLWQDDIDNLAVPPTPA
QETAAAPADAALLAALAPLDAARRQQHLVQWLAARAAAALGTVAARAIRP
EASLFGYGLDSMSATRLAAVAAAASGLALPDSLLFDHPSLDGLAGWLLQA
MEQARHLPPAPGGRDRAMPAPRPAAHRHGDGQDPIAIIGMAFRLPGENGH
DADTDAAFWRLLDGAGCAIRPMPAERFRAPAGMPGFGAYLNQVDRFDAAF
FGMSPREAMNTDPQQRLLLEVAWHALEDAGLPPGDLRGSDSGVFVGIGTA
DYGHLPFISGDDAHFDAYWGTGTSFAAACGRLSFTFGWEGPSMAVDTACS
ASHSALHLAVQALRARECGMALSAGVKLQLLPEIDRVLHKAGMLAADGRC
KTLDASADGYVRGEGCVVLVLKRLSDALADGDAIRAVIRDTLVRQDGAGS
SLSAPNGEAQQRLLSLALARAGLAPSEIDYIELHGTGTRLGDPIEYQSVA
DVFGGRAPDDPLWIGSVKTNIGHLESAAGAAGLVKTVLALEQARIPPLVG
LKGINPLIDLDAIPARAPAHTVDWPARQAVRRAGVTSYGFAGTIAHVILE
QAPQAPVAQAAGTEPTRGPHLFLLSARSPDALRRLAAAYRDTLAGTADLA
VLANGMARQREHHALRAAVVASDHDECARALDRLAAPDAAAPEAVTRAPR
VGFLFTGQGSQYAGMTRALYAAQPDFRAALDAADAALAPHLGRSILALMH
DDAQRDALQQTAHAQPALFACGYALAAMWQAWGVVPAVLVGHSIGEFAAM
VVAGAMTLEDAARLIVRRGALMQALPAGGAMLAARATPRHAHDLLAALAP
AVAAEVSLAAINGPQDVVFSGSAAGIDAVRARLDAQQLDARPLAVSHAFH
SPLLDPMLGDWAEACADAQSAPPRIPLISTLTGAPMTTAPDAAYWSAHAR
QPVRFAEALARAGADCDVLLEIGAHAVLSALAQRNQLAQPWPHPVACVAS
LLRGTDDSRAVAQACAELYLRGQPFDWDRLFAGPLPSPRALPRYPFDRQS
HWLEYDEDAPRTPLPMQPQPERAAPRPVERYAVQWEPFAPSAGDGHASTY
WIVAADAADAGPADAGRLAARLSGPARDVHVLSPSQWADAADRIADDDVV
IYLAGWPARASDAAAVAGSRHVWQLTECVRTLQRLRKTPRILLPTLHGQS
PDGAPCDPLQAALWGAARPLSLEYPGPAWLLADCAGESPLETLADALPAL
LPLFGKEEAVALRAGGWLRPRLTPQAAPERAPCVTLRADGLYLVAGAYGA
LGRHTTDWLAAHGATHLVLAGRRAPPAGWQARLALLRAQGVRIDPVDADL
AEAADVERLFDAVAALEATTGRTLAGVFHCAGTSRFNDLAGLTTDDCAAV
TGAKMTGAWLLHEQTRARRLDWFVCFTSISGVWGSRLQIPYGAANAFQDA
LVRLRRAQGLPALAVAWGPWGGGAGMSEVDDALLQLLRAAGIRRLAPSRY
LATLDHLLGHAEHADGLPADGTCVVAEVDWQQFIPLFALYNPIGTFERCR
TDTATHATAAPSALIALDSGARADAVRAFVIAELARTLRVAPSQLTPDIE
LLKLGMDSILVMDFSRRCESGLGVKCELKAIFERNTPGGLASYLLERLEH
APQGAVPAPAAAEPIVHAPDHAHLPFPLTELQHAYWIGRQGHYALGGVAC
HAYLEADAADGLDLGLLERCWNALVARHGALRLVIDESGQQRILPRVPAY
RIRVANLGAATPQALAAHCDDWRQAMSHQVLDAAQWPLFDVRATHLPGGA
TRLHIGIDMLINDATSGQIIWDELAALYRAGGDLERAGLAPFEISFRDYV
LAKYVHSEARRAARESAKAYWLGQLETLPPAPQLPLRAEALHRAAPRFSR
RQHRLSAPQWQSLRDRAAASGCTPASLLIAVFAEVLSAWSTEPRFTLNLT
TFDRLPWHADVPRLLGDFTAVTLLPLDCAAPLPFGQRAAAVNGAVLEHLQ
HRAFSAVDVLREWNRGRERQDAVSMPVVFTSQLGMSDPTKGAARASVLGT
VGYGISQTPQVWLDHQACELDGALIYNWDAVDALFQPGVLDAMFDAYNRM
LERLAADADAWLEPLPALLPQAQREVRARVNASTAPLPERCLDQLFFDQA
Truncated Ralstonia solanacearum MicA
amino acid sequence encoded by Ralstonia
solanacearum micA gene; 832 amino acids
in length.
SEQ ID NO: 2
MMTITTDRTPPAAGAALDRNRSAYAGLADVLERAGLAEHALYLNWGYRPV
DGQPDWAARELPPGELGRMQARLVLEVLGDTPLDGRRVLDVGCGRGGALA
LMGRLHAPAALAGADISAANIAYCRKRHTHPRLRFQIADACRLPYPDSSM
DVVFNLESSGAYPDIGAFFHHVHRILRVGGRFCLADVFDADSVAWVRAAL
EQAGFTLERERSIPAQVRAARERASPGIWRRLDTALTALDAPGLRRELER
YLAAPSSGLFQALEDGRVDYRLFHWRKTCPAAGRIDADVIARLATRSARL
DAALQDRAPSAAAPQSPAPGPANASASAWFPFTAPDAQAGFNVFALPYAG
GGASVYRAWTLPRRPGAAPWQLCPVQLPGRESRFGEPLIDDMATLADRLA
DAIGPYAHRPWALLGCSLGCKIAFEVARRFARQGRPPALLFLMACPAPGL
PLGRRISTRAEADFAREVCHLGGTPPEVLADAEMMRTLMPILRNDSALAE
HYVAAEDATVNVPIVMVAAGDDHLVTVEEARRWQRHAGAGFDWRLVDGGH
FFLRQRRRELTDWLLDALRRGERTLPVQTTTTDVPDVPCSTPEQPRDPSR
MPAPGASANLVLAPGEILVVTAPRSLAARLTPAVLSDDEQRQLARFAFDA
DRERYLAAHWAKRRVLGALLAAAPRSLRFGAQAGGKPYLIGEALHFSLSH
SGDRVAVAVCRHAPVGVDIEQARGIACHASAARIMHPLDRIAPQCETPED
RFLAAWSLKEAVAKCTGAGLALPFDSLRLAFAGNGRYGCLLGTHAAWEAH
HQHEDGVHLAVASATPWAALRILPLDAALAEG
Streptomyces sp. A2991200 BenA amino
acid sequence without the signal peptide
sequence from amino acids 2-29 encoded
by the Streptomyces BenA gene
SEQ ID NO: 3
MAGRTATRRITLFDPERFRCRIAAECDFDAAALGLTPQEIRRMDRAVQMA
VAATGEALADAGVGEGDLDPARTGVTIGNAVGSTMMMEEEYVVISDGGRK
WLCDEEYGVRHLYGAVIPSTAGVEVARRVGAEGPTAVVSTGCTSGLDAVG
HAAQLIEEGSADVVIGGATDAPISPITVACFDSLKATSTRNDDAEHACRP
FDRDRDGLVLGEGSAVFVMEARERAVRRGAKIYCEVAGYAGRANAYHMTG
LKPDGRELAEAIDRAMAQAGISAEDIDYVNAHGSGTRQNDRHETAAFKRS
LRDHARRVPVSSIKSMVGHSLGAIGAIEVAASALAIEHGVVPPTANLTTP
DPECDLDYVPREAREHPTDVVLSVGSGFGGFQSAVVLISPRSRR
Streptomyces sp. A2991200 BenQ amino
acid sequence
SEQ ID NO: 4
MSQLSLSQAAPAGGSRIRGVGAYRPARVVTNEEIAPRIGVAPEWIARRSG
IHTRRFAGPDEPLAMMAATASEKALAAAGLSADEVDCVLVATISHLLQMP
ALAVDVAHRLGAAPTAAFDLSAACAGFCHGVAIADSMVRSGTAHNVLLVG
ADRMTDVVDADDPATAFLFADGAGAVVIGPSETPGIGPVAWGSDGERMDA
ITMTGHWTPSLRTNPELPWPYLCMTGWKVFRWATETMGQAARDAIERAGV
TSEELSAFIPHQANGLITDALAKDIGLTADTAIARDITDSGNTSGASIPM
AMERLLASGQARSGEAALLIGFGSGLVHAGQVVLLP
RevS polypeptide sequence GenBank BAK64635.1
SEQ ID NO: 5
MELALPAELAPTLPEALRLRSEQQPDTVAYVFLRDGETPEETLTYGRLDR
AARARAAALEAAGLAGGTAVLLYPSGLEFVAALLGCMYAGTAGAPVQVPT
RRRGMERARRIADDAGAKTILTTTAVKREVEEHFADLLTGLTVIDTESLP
DVPDDAPAVRLPGPDDVALLQYTSGSTGDPKGVEVTHANFRANVAETVEL
WPVRSDGTVVNWLPLFHDMGLMFGVVMPLFTGVPAYLMAPQSFIRRPARW
LEAISRFRGTHAAAPSFAYELCVRSVADTGLPAGLDLSSWRVAVNGAEPV
RWTAVADFTEAYAPAGFRPQAMCPGYGLAENTLKLSGSPEDRPPTLLRAD
AAALQDGRVVPLTGPGTDGVRLVGSGVTVPSSRVAVVDPGTGTEQPAGRV
GEIWINGPCVARGYHGRPAESAESFGARIAGQEARGTWLRTGDLGFLHDG
EVFVAGRLKDVVIHQGRNFYPQDIELSAEVSDRALHPNCAAAFALDDGRT
ERLVLLVEADGRALRNGGADALRARVHDAVWDRQRLRIDEIVLLRRGALP
KTSSGKVQRRLARSRYLDGEFGPAPAREA
Illustrative Cannabis sativa CSAAE3
polypeptide sequence; GenBank AFD33347.1
SEQ ID NO: 6
MEKSGYGRDGIYRSLRPPLHLPNNNNLSMVSFLFRNSSSYPQKPALIDSE
TNQILSFSHFKSTVIKVSHGFLNLGIKKNDVVLIYAPNSIHFPVCFLGII
ASGAIATTSNPLYTVSELSKQVKDSNPKLIITVPQLLEKVKGFNLPTILI
GPDSEQESSSDKVMTFNDLVNLGGSSGSEFPIVDDFKQSDTAALLYSSGT
TGMSKGVVLTHKNFIASSLMVTMEQDLVGEMDNVFLCFLPMFHVFGLATT
TYAQLQRGNTVISMARFDLEKMLKDVEKYKVTHLWVVPPVILALSKNSMV
KKFNLSSIKYIGSGAAPLGKDLMEECSKVVPYGIVAQGYGMTETCGIVSM
EDIRGGKRNSGSAGMLASGVEAQIVSVDTLKPLPPNQLGEIWVKGPNMMQ
GYFNNPQATKLTIDKKGWVHTGDLGYFDEDGHLYVVDRIKELIKYKGFQV
APAELEGLLVSHPEILDAVVIPFPDAEAGEVPVAYVVRSPNSSLTENDVK
KFIAGQVASFKRLRKVTFINSVPKSASGKILRRELIQKVRSNM
Illustrative Cannabis sativa CSAAE1
polypeptide sequence; GenBank AFD33345.1
A transmembrane domain that is optionally
removed is underlined.
SEQ ID NO: 7
MGKNYKSLDSVVASDFIALGITSEVAETLHGRLAEIVCNYGAATPQTWIN
IANHILSPDLPFSLHQMLFYGCYKDFGPAPPAWIPDPEKVKSTNLGALLE
KRGKEFLGVKYKDPISSFSHFQEFSVRNPEVYWRTVLMDEMKISFSKDPE
CILRRDDINNPGGSEWLPGGYLNSAKNCLNVNSNKKLNDTMIVWRDEGND
DLPLNKLTLDQLRKRVWLVGYALEEMGLEKGCAIAIDMPMHVDAVVIYLA
IVLAGYVVVSIADSFSAPEISTRLRLSKAKAIFTQDHIIRGKKRIPLYSR
VVEAKSPMAIVIPCSGSNIGAELRDGDISWDYFLERAKEFKNCEFTAREQ
PVDAYTNILFSSGTTGEPKAIPWTQATPLKAAADGWSHLDIRKGDVIVWP
TNLGWMMGPWLVYASLLNGASIALYNGSPLVSGFAKFVQDAKVTMLGVVP
SIVRSWKSTNCVSGYDWSTIRCFSSSGEASNVDEYLWLMGRANYKPVIEM
CGGTEIGGAFSAGSFLQAQSLSSFSSQCMGCTLYILDKNGYPMPKNKPGI
GELALGPVMFGASKTLLNGNHHDVYFKGMPTLNGEVLRRHGDIFELTSNG
YYHAHGRADDTMNIGGIKISSIEIERVCNEVDDRVFETTAIGVPPLGGGP
EQLVIFFVLKDSNDTTIDLNQLRLSFNLGLQKKLNPLFKVTRVVPLSSLP
RTATNKIMRRVLRQQFSHFE
Illustrative olive tolic acid cyclase
polypeptide sequence; UniProtKB/Swiss-Prot:
16WU39.1
SEQ ID NO: 8
MAVKHLIVLKFKDEITEAQKEEFFKTYVNLVNIIPAMKDVYWGKDVTQKN
KEEGYTHIVEVTFESVETIQDYIIHPAHVGFGDVYRSFWEKLLIFDYTPR
K
olive tolic acid cyclase polypeptide
sequence lacking the N-terminal
methionine and C-terminal lysine
relative to SEQ ID NO: 5
SEQ ID NO: 9
AVKHLIVLKFKDEITEAQKEEFFKTYVNLVNIIPAMKDVYWGKDVTQKNK
EEGYTHIVEVTFESVETIQDYIIHPAHVGFGDVYRSFWEKLLIFDYTPR
Truncated version of cyclase, 95 aa,
lacking the N-terminal me thionine
and five amino acid sequence YTPRK
(SEQ ID NO: 22) at the C-terminal
end relative to SEQ ID NO: 5
SEQ ID NO: 10
AVKHLIVLKFKDEITEAQKEEFFKTYVNLVNIIPAMKDVYWGKDVTQKNK
EEGYTHIVEVTFESVETIQDYIIHPAHVGFGDVYRSFWEKLLIFD
Amino acid sequence of 415-amino acid
C-terminal domain of Ralstonia
solanacearum acyl-CoA synthase
SEQ ID NO: 11
MAFNERVVDWQQVAGAQPDASPERMSADDPFMIIYTSGTTGKPKGTVHTH
GSFPMKIAHDSAIHFNVSPKDVFCWPADMGWVAGTLVMSCALLRGATLVC
YDGAPDFPDWSRMSRLIERHRVTHFGSAPTLIRGLASNEAIATQGDVSSV
KLLITAGEGIDPEHFLWFQKAFGGGHRPVINYTGGTEVSGALLSSVVIKP
ISPAGFNTASPGVATDVVDAEGHSVTGEVGELAIRKPFIGMTRSFWQDDE
RYLDSYWRTIPGIWVHGDLAMRREDGMWFMMGRSDDTIKLAGKRLGPAEI
EDVLLELPEIAEAAAIGVEDPVKGQKLVVFVVASKASTASADALASVIGK
HVDLRLGRPFRPSVVHVVAQLPKTRSSKIMRRVIRSVYTGKPAGDLSSLD
NPLALDEIRSAAAVS
Amino acid sequence of Arabidopsis
thaliana AtHS1 cyclase
SEQ ID NO: 12
MEEAKGPVKHVLLASFKDGVSPEKIEELIKGYANLVNLIEPMKAFHWGKD
VSIENLHQGYTHIFESTFESKEAVAEYIAHPAHVEFATIFLGSLDKVLVI
DYKPTSVSL
Amino acid sequence of N-terminal
domain of BenH polypeptide from
Streptomyces sp. A2991200
SEQ ID NO: 13
AGRTDNSVVIDAPVQLVWDMTNDVSQWAVLFEEYAESEVLAVDGDTVRFR
LTTQPDEDGKQWSWVSERTRDLENRTVTARRLDNGLFEYMNIRWEYTEGP
DGVRMRWIQEFSMKPSAPVDDSGAEDHLNRQTVKEMARIKKLIEEA
Aspergillus nidulans orsA; First
216 aa SAT domain
SEQ ID NO: 14
MAPNHVLFFPQERVTFDAVHDLNVRSKSRRRLQSLLAAASNVVQHWTASL
DGLERADIFSFEDLVELAERQTTQTRGSIVADLVLLTTVQIGQLLVLAED
DPAILSGHAGARAIPMGFGAGLVAAGVAAAATSADGIVNLGLEAVSVAFR
LGVELQRRGKDIEDSNGPWAQVISSATTIADLEQALDRINASLRPINQAY
IGEVMTESTVVFGPPS
Fusarium graminearum PKS14 (OSAS)
2373 aa
SEQ ID NO: 15
MAARRVVLFGGQGSRSIFSSSTTSIAEQDAQSSTAGILLSKCHVAILREI
SSLDVQSRLILAIDPVSFPTPRHLLQIADKYHTHPVLQATTIYLCQILRY
LSHTLQQDDTFEQCFERIEATAGFSSGIIPAAVVACSSTIDEFVVCAVEG
FRLAFWVAYYSFRWSLLLAEQNGHNTSQDATMSLATRGLSRTQVEQVLYR
MKAERGLQRMAISSIAISGSVSISGPQAELVALQGELQSLRYVTTTFAYV
HGWYHGGKQLEPVVKQVEETINRRCICFPSCDGSSKPIYSTLDGTVLDLF
GGSSNKPLSSLTRHLLIHCVNWRDTSRAIAADIREILRHTPMAVDILSFG
PASSSIFPTIDSQNPRVNLVDMSSFKSQEGSTTQHLDRPNDIAIVGMSTN
LPGGHNAAQLWETLSSGLNTVQEIPESRFQISDYYTSEKGEPRSMATGHG
AFLDDPFSFDNAFFNISPREAKSMDPQQRILLHGAQEALEDAGYVADSTP
SSQRATTGCYIGLATGDYTDNLHDDIDAFYPSGTLRAFHSGRISYFYQLS
GPSIVTDTACSSSTVSIYQACRAIQNGDCTTAIAGGVNVITSPDMYLSLS
RGHFLSPTGNCKPFDASADGYCRAEGCVLFVLKRLSDAVAEGDRIHAVIR
NAQINQSGNSSSITHPHSPTQTDLLTRLLKQADVDPASISVVEAHGTGTQ
AGDAREIETLKLVFSQYHSATTPLVVSSIKGNVGHCEAASGAAGLAKLLL
MLRNDEIPKQAGLENMNPALGDLQNSGLVVPRQNMPWNRSRTVPRRAVLN
NFGAAGSNASLLLEEWLESPATSKQKNEEGKRSSYVFALSAKSNKALQLS
VGRHIETLKKNMELGTSLEDICYTATARRQQFDHRISATCSSKLELMDKL
EQYQSTVSTPAQMVSSTVFIFTGQGSIYSGMGRELMSTYPPFRDIIRTCD
RIVQGLGLGCPSILNYILPGTEGRLASMSHVEHLMVSQCACVALEYALAK
TFISWGIKPDYVMGHSLGEYTALCISGVLTPGDTFRLVATRAKMMGEHCA
ANTSGMLACHLSSGEIQSIISDDPSFCQLSIACLNGPHDCVVGGPLTQLE
ALRTRCKTGNIKCKLIDVPYAFHTSAMDPVLGLLSALGRSVEFQDATIPV
ISNVDGQLFRKDMTANYFANHTRRPVRFHESIMNLQDLIGQSSLDESLFI
EIGPQPAMLPMLRDSIASASCTYLSTLQKGRDAWMSISETLSAISLRKMG
INWREVFDGTSAQVTDLPGHPLQGTRFCIPFKEPRGITNHAKSSAIAFAT
SVRTGCRLLPWVRADTNLSKEHIFETDMTTLGPLISGHDVGGSPICPASV
FHELALEAAKSVLEPGKEDILVVKGMKFSSPLIFLSSTSNTTVHVHISKK
GIATTRTASFHVKSTSPASPVESLHCSGYVTLQNLEQQSGQWMRDHALVT
RQARLFSGAGKDLLSTFRRRVLYENIFTRVVRYSRDYQTLQFLDVADSNL
EGMGSFNMPSDSIAQTETAYIAHPVFTDTLLHAAGFIANLAIGSNEVGIC
SAVESIEVAYHEINYEDTFKIYCSLLEVKGLIVADSFALDSSDNIVAVIR
GMEFKKLQLSTFQQALSRISSNSEPEGPEYHHGVSSSAELQLQTSVAACQ
PLTVDTAIDAHKHQDENGISQILKDVVVEVGGFMEQDIDYTMSLTSLGID
SLMQIEIVSKISRLFPEKTGLDHNALAECETLQELNDMLSSVLQPSVKQR
SASQASSSKQTAVITPTSSDSSVEGDSAHGSVVLPVALHTSDESRTPLCL
FHDGSGQISMYKRLQGHDRTTYAFFDPKFECSDEGRSFYSSIEDMAEDYA
SRILSTRPPLSSLILCGWSFGGIVALEVARLLFLRGIEVRGLVLIDSPSP
INHEPLPAQIISSITRFTGRSESTNALEEEFLSNASLLGRYKPESLSLTT
GRTLKTVMLQSKGTLDTESLCGVRYDWLSRQDVRDAAIAEWESLMTRSPK
REIHNFGKHANTSNSLTDKSSASNKAHISMHQRIDLHCHAVAPSYRQYAI
DNGHEKPDGMPALPQWTPEQHIGLMKKLNISKSVLSITSPGTHLTPQNDE
NATRLTRQVNEELSTICQKHPSYFSFFASLPLPSVNDSIAEIDYALDQLG
ALGFAVLSNANGVYLGDAELDPVFAHLNARKAILFIHPTTCNIIASSGQV
QPVKPLEKYPRPMMEFMFDETRAIANLLLSGTVAKYPDIKFIMSHCGCAL
PSMLDRIGAFATLISGAESQTAEFQRLLRERFYFDLAGFPLPNAIHGLLR
ILGEGAEKRLVYGTDYPFTPERLVVSLADVMEKGLEELFDEGQRADVLVR
VAGTIQDEAMRTTNTEDHSGTLS
full-length BenA 423 aa
SEQ ID NO: 16
MSSERRAVITGMGVIAPGGVGTRAFWSAVTAGRTATRRITLFDPERFRCR
IAAECDFDAAALGLTPQEIRRMDRAVQMAVAATGEALADAGVGEGDLDPA
RTGVTIGNAVGSTMMMEEEYVVISDGGRKWLCDEEYGVRHLYGAVIPSTA
GVEVARRVGAEGPTAVVSTGCTSGLDAVGHAAQLIEEGSADVVIGGATDA
PISPITVACFDSLKATSTRNDDAEHACRPFDRDRDGLVLGEGSAVFVMEA
RERAVRRGAKIYCEVAGYAGRANAYHMTGLKPDGRELAEAIDRAMAQAGI
SAEDIDYVNAHGSGTRQNDRHETAAFKRSLRDHARRVPVSSIKSMVGHSL
GAIGAIEVAASALAIEHGVVPPTANLTTPDPECDLDYVPREAREHPTDVV
LSVGSGFGGFQSAVVLISPRSRR
BenB 409 aa
SEQ ID NO: 17
MTVITGLGVVAPTGVGLDDYWATTLAGKSGIDRIRRFDPSGYTAQLAGQV
DDFEATDHVPSKLLAQTDRMTHFAFAGANMALADAHVDLADFPEYERAVV
TANSSGGVEYGQHELQKMWSGGPMRVSAYMSVAWFYAATTGQLSIHHGLR
GPCGLIATEQAGGLDALGHARRLLRRGARIAVTGGTDAPLSPASMVAQLA
TGLLSSNPDPTAAYLPFDDRAAGYVPGEGGAIMIMEPAEHALRRGAERIY
GEIAGYAATFDPAPGTGRGPTLGRAIRNALDDARIAPSEVDLVFADGSGT
PAMDRAEAEALTEVFGPRGVPVTVPKAATGRMYSGGGALDVATALLAMRD
GVAPPTPHVTELASDCPLDLVRTEPRELPIRHALVCARGVGGFNAALVLR
RGDLTTPEH
BenC
SEQ ID NO: 18
MSTLSVEKLLEIMRATQGESADTSGLTEDVLDKPFTDLNVDSLAVLEVVT
QIQDEFKLRIPDSAMEGMETPRQVLDYVNERLEEAA
Full-length B enH; the truncated version
SEQ ID NO: 13 is underlined.
SEQ ID NO: 19
MAGRTDNSVVIDAPVQLVWDMTNDVSQWAVLFEEYAESEVLAVDGDTVRF
RLTTQPDEDGKQWSWVSERTRDLENRTVTARRLDNGLFEYMNIRWEYTEG
PDGVRMRWIQEFSMKPSAPVDDSGAEDHLNRQTVKEMARIKKLIEEAAAR
AGVDGGIPAEGKDSVRDATGNGDPGPVFRVLLRAEIADGKEKEFEDAWRE
IGQVITGQPANLGQWLMRSHDEPGVYYIISDWTDEERFRAFERSEEHVGH
RSTLQPFRTKGSMVTTDVVAAMTKAGQTW
A. nidulans orsA; 2103 aa
SEQ ID NO: 20
MAPNHVLFFPQERVTFDAVHDLNVRSKSRRRLQSLLAAASNVVQHWTASL
DGLERADIFSFEDLVELAERQTTQTRGSIVADLVLLTTVQIGQLLVLAED
DPAILSGHAGARAIPMGFGAGLVAAGVAAAATSADGIVNLGLEAVSVAFR
LGVELQRRGKDIEDSNGPWAQVISSATTIADLEQALDRINASLRPINQAY
IGEVMTESTVVFGPPSTLDALAKRPELAHATITSPASALAQVPLHGAHLP
PISATMIAASSSQQATELWKLAVEEVANKPIDVHQAVTALIHDLHRANIT
DIVLTAIGASTETSGIQSLLEKNGLAVELGQLSPTPRPYGNDLDSIPADA
IAVVGMSGRFPNSDTLDEFWRLLETATTTHQVIPESRFNVDDFYDPTRAK
HNALLARYGCFLKNPGDFDHRLFNISPREAMQMDPVQRMLLMTTYEALEM
AGYSPPTPAAPGDSEQAPPRIATYFGQTIDDWKSINDQQGIDTHYLPGVN
RGFAPGRLSHFFQWAGGFYSIDTGCSSSATALCLARDALTAGKYDAAVVG
GGTLLTAPEWFAGLSQGGFLSPTGACKTYSDSADGYCRGEGVGVVILKRL
ADAVRSKDNVIAVIAGASRNCNAGAGSITYPGEKAQGALYRRVMRQAAVR
PEQVDVVEMEIGTGTQAGDRVETHAVQSVFAPSNGNQREKPLIVGALKAN
IGHSEAAAGIISLMKAILILQHDKIPAQPNQPIKMNPYLEPLIGKQIQLA
NGQSWTRNGAEPRYIFVNNFDAAGGNVSMLLQDPPAFALPAPASGPGLRT
HHVVVTSGRTATAHEANRKRLHAYLSAHPDTNLADLAYTTTARRIHNVHR
EAYVASSTSDLVRQLEKPLADKVESAPPPAVVFTFTGQGAQSLGMGGALY
STSPTFRRLLDSLQSICEVQGLPTKFLNAIRGSGAEGATVTEVDMQVATV
ALEIALARYWRSLGIRPTVLIGHSLGEYAALCVAGVLSASDALALAFRRA
TLIFTRCPPSEAAMLAVGLPMRTVQYRIRDSAATTGCEVCCVNGPSSTVV
GGPVAAIQALDEYLKSDGKVSTTRLRVQHAFHTRQMDVLLDELEASAAQV
PFHAPTLPVASTVLGRIVRPGEQGVFDANYLRRHTREPVAFLDAVRACET
EGLIPDRSFAVEIGPHPICISLMATCLQSAKINAWPSLRRGGDDWQSVSS
TLAAAHSAQLPVAWSEFHKDHLDTVRLISDLPTYAFDLKTFWHSYKTPAA
AVSAASATPSTTGLSRLASTTLHAVEKLQREEGKILGTFTVDLSDPKLAK
AICGHVVDESAICPASIFIDMAYTAAVFLEQENGAGAALNTYELSSLEMH
SPLVLREDIEVLPQVWVEAVLDIKSNAVSVHFKGQTSKGAVGYGSATMRL
GQPDSAVRRDWSRIQSLVRARVQTLNRSVRPREVHAMDTALFYKVFSEIV
DYSAPYHAVQEAVIAADFHDAAVTLQLTPTADLGTFTSSPFAVDALVHVA
GFLLNADVRRPKNEVHIANHIGSLRIVGDLSSPGPYHVYATIREQDQKAG
TSLCDVYTTDSQDRLVAVCSDICFKKLERDFFALLTGATRGRSTKPVAAA
PAKSMAKRARQLAPSPSPSSSSGSNTPMSRSPTPSSVSDMVDLGTELLQA
VAEQTGVSVAEMKSSPGTTFTEFGVDSQMAISILANFQRTTAVELPAAFF
TNFPTPADAEAELGGSALDDLEEDITKPTPSPEQTQARKQGPAPSQHLLS
LVAQALGLEASDLTPSTTFDSVGMDSMLSIKITAAFHAKTGIELPAAFFS
ANPTVGAAQEALDDDAEEESAPAQTSTNPAKETTIDSSRQHKLDAAVSRA
SYIHLKALPKGRRIYALESPFLEQPELFDLSIEEMATIFLRTIRRIQPHG
PYLIGGWSAGSMYAYEVAHRLTREGETIQALIILDMRAPSLIPTSIVTTD
FVDKLGTFEGINRARDLPEDLSVKERAHLMATCRALSRYDAPAFPSDRQP
KQVAVVWALLGLDNRPDAPIASMGRPGLDIGKSMYEMNLDEFERYFNSWF
YGRRQQFGTNGWEDLLGDHIAVYTVNGDHFSMMCPPYASEVGDIVIETVT
RAVE
olivetolic acid synthase polypeptide
sequence; UniProtKB/Swiss-P rot:
B1Q2B6. 1
SEQ ID NO: 21
MNHLRAEGPASVLAIGTANPENILLQDEEPDYYFRVTKSEHMTQLKEKFR
KICDKSMIRKRNCFLNEEHLKQNPRLVEHEMQTLDARQDMLVVEVPKLGK
DACAKAIKEWGQPKSKITHLIFTSASTTD1VfPGADYHCAKLLGLSPSVK
RVMMYQLGCYGGGTVLRIAKDIAENNKGARVLAVCCDIMACLFRGPSESD
LELLVGQAIFGDGAAAVIVGAEPDESVGERPIFELVSTGQTILPNSEGTI
GGHIREAGLIFDLHKDVPMLISNNIEKCLIEAFTPIGISDWNSIFWITHP
GGKAILDKVEEKLHLKSDKFVDSRHVLSEHGNMSSSTVLFVMDELRKRSL
EEGKSTTGDGFEWGVLFGFGPGLTVERVVVRSVPIKY

1. A modified recombinant host cell comprising: (i) a first exogenous polynucleotide that encodes a BenA polypeptide comprising an amino acid sequence having at least 95% identity to SEQ ID NO:16 (ii) a second exogenous polynucleotide that encodes a BenB polypeptide comprising an amino acid sequence having at least 95% identity to SEQ ID NO:17, (iii) a third exogenous polynucleotide that encodes a BenC polypeptide comprising an amino acid sequence having at least 95% amino acid identity to SEQ ID NO:18; and (iv) a fourth exogenous polynucleotide comprising an amino acid sequence that encodes an N-terminal domain of a BenH polypeptide, wherein the N-terminal domain of the BenH comprises an amino acid sequence having at least 95% identity to SEQ ID NO:13.

2.-6. (canceled)

7. The modified recombinant host cell of claim 1, wherein one or more of the exogenous polynucleotides are integrated into the host genome.

8.-9. (canceled)

10. The modified recombinant host cell of claim 1, wherein the host cell is a cell selected from the group consisting of a Saccharomyces cerevisiae, Kluyveromyces lactis, Kluyveromyces marxianus, Pichia pastoris, Yarrowia lipolytica, Hansenula polymorpha and Aspergillus cell.

11. A method of producing a cannabinoid product or a cannabinoid precursor product, the method comprising culturing a modified recombinant host cell of claim 1 under conditions in which the exogenous polynucleotides are expressed, thereby producing the cannabinoid product or cannabinoid precursor product.

12. The method of claim 11,
wherein the modified recombinant host cell is cultured under conditions in which products encoded by the exogenous polynucleotides are expressed and a 5-alkyl-benzene-1,3-diol is produced; and
converting the 5-alkyl-benzene-1,3-diol to the cannabinoid product.

13.-14. (canceled)

15. A modified recombinant host cell comprising: (i) a first exogenous polynucleotide that encodes an acyl-CoA synthetase that converts an aliphatic carboxylic acid to an acyl CoA thioester, (ii) a second exogenous polynucleotide that encodes a Type II polyketide synthase (PKS), wherein the Type II PKS is a BenA PKS that comprises BenA, BenB, and BenC polypeptide; (iii) and a third exogenous polynucleotide that encodes a 2-alkyl-4,6-dihydroxybenzoic acid cyclase.

16. The modified recombinant host cell of claim 15, wherein the aliphatic carboxylic acid is hexanoic acid.

17. (canceled)

18. The modified recombinant host cell of claim 15, further comprising an exogenous polynucleotide encoding a BenQ polypeptide.

19. The modified recombinant host cell of claim 15, wherein the 2-alkyl-4,6-dihydroxybenzoic acid cyclase is olivetolic acid cyclase or a truncated olivetolic acid cyclase, an AtHS1 polypeptide, or the N-terminal domain of a BenH polypeptide; and/or the acyl-CoA synthetase is a revS polypeptide, a CsAAE3 polypeptide, or a transmembrane domain-deleted CsAAE1 polypeptide.

20.-21. (canceled)

22. The modified recombinant host cell of claim 15, further comprising an exogenous polynucleotide that encodes a prenyltransferase that catalyzes coupling of geranyl-pyrophsophate to a 2-alkyl-4,6-dihydroxybenzoic acid to produce an acidic cannabinoid.

23. The modified recombinant host cell of claim 15, wherein the modified recombinant host cell is a yeast cell genetically modified to knockout expression of the PAD1 and FDC1 aromatic decarboxylase genes.

24.-30. (canceled)

31. A modified recombinant host cell comprising: (i) a first exogenous polynucleotide that encodes an acyl-CoA synthetase that converts an aliphatic carboxylic acid to an acyl CoA thioester, (ii) a second exogenous polynucleotide that encodes a Type I polyketide synthase (PKS), wherein the type I PKS is a MicC PKS from the bacterium Ralstonia solanacearum, (iii) and a third exogenous polynucleotide that encodes a 2-alkyl-4,6-dihydroxybenzoic acid cyclase.

32. (canceled)

33. The modified recombinant host cell of claim 31, wherein the host cell further comprises an exogenous polynucleotide encoding MicA from the bacterium Ralstonia solanacearum.

34. The modified recombinant host cell of claim 31, wherein the aliphatic carboxylic acid is hexanoic acid or butanoic acid.

35. The modified recombinant host cell of claim 31, wherein the 2-alkyl-4,6-dihydroxybenzoic acid cyclase is olivetolic acid cyclase, a truncated olivetolic acid cyclase, an AtHS1 polypeptide, or the N-terminal domain of a BenH polypeptide; and/or the acyl-CoA synthetase is a revS polypeptide, a CsAAE3, or a transmembrane domain-deleted CsAAE1.

36.-37. (canceled)

38. The modified recombinant host cell of claim 31, further comprising an exogenous polynucleotide that encodes a prenyltransferase that catalyzes coupling of geranyl-pyrophsophate to a 2-alkyl-4,6-dihydroxybenzoic acid to produce an acidic cannabinoid.

39.-45. (canceled)

46. A method of producing a cannabinoid product, the method comprising culturing a modified recombinant host cell of claim 31 under conditions in which products encoded by the exogenous polynucleotides are expressed and a 2-alkyl-4,6-dihydroxybenzoic acid or 5-alkyl-benzene-1,3-diol is produced; and
converting the 2-alkyl-4,6-dihydroxybenzoic acid or 5-alkyl-benzene-1,3-diol to the cannabinoid product.

47.-51. (canceled)

52. A method of producing a cannabinoid or cannabinoid precursor product, the method comprising culturing a modified recombinant host cell of claim 15 under conditions in which the cannabinoid or cannabinoid precursor is produced.

53. The method of claim 52, wherein the aliphatic carboxylic acid is hexanoic acid.

54. (canceled)

55. The method of claim 52, wherein the modified recombinant host cell further comprises an exogenous polynucleotide encoding a BenQ polypeptide.

56.-58. (canceled)

59. The method of claim 52, wherein the modified recombinant host cell further comprises an exogenous polynucleotide that encodes a prenyltransferase that catalyzes coupling of geranyl-pyrophsophate to a 2-alkyl-4,6-dihydroxybenzoic acid to produce an acidic cannabinoid.

60. (canceled)

61. The method of claim 52, wherein the 2-alkyl-4,6-dihydroxybenzoic acid or 5-alkyl-benzene-1,3-diol is the cannabinoid precursor product.

62. The method of claim 61, further comprising converting the 2-alkyl-4,6-dihydroxybenzoic acid or 5-alkyl-benzene-1,3-diol to the cannabinoid product.

63.-69. (canceled)
